(19) United States
(12) Patent Application Publication
(10) Pub. No.: US 2017/0211102 A1
(43) Pub. Date: Jul. 27, 2017

(54) METHODS FOR BIOLOGICAL PRODUCTION OF VERY LONG CARBON CHAIN COMPOUNDS

(71) Applicant: CALYSTA, INC., Menlo Park, CA (US)

(72) Inventors: Lorraine Joan GIVER, Sunnyvale, CA (US); Joshua A. SILVERMAN, Los Altos Hills, CA (US); John H GRATE, Los Altos, CA (US)

(21) Appl. No.: 15/311,080

(22) PCT Filed: May 14, 2015
(86) PCT No.: PCT/US2015/030836

§ 371 Date: Nov. 14, 2016

Related U.S. Application Data
(60) Provisional application No. 61/994,042, filed on May 15, 2014.

(51) Int. Cl. C12P 11/00; C12N 1/20; C12N 15/52

(52) U.S. Cl. CPC C12P 11/00; C12N 15/52; C12N 1/20

(57) ABSTRACT

The present disclosure provides compositions and methods for biologically producing very long carbon chain compounds (longer than C₂₄), such as fatty acyl-CoA, fatty aldehydes, fatty alcohols, fatty ester waxes, alkanes and ketones, from recombinant C₁ metabolizing microorganisms that utilize C₁ substrates, such as methane or natural gas as a feedstock.

### BACKGROUND

Technical Field

The present disclosure provides compositions and methods for biologically producing very long chain carbon compounds and, more specifically, using recombinant C₁ metabolizing microorganisms to produce very long chain fatty alcohols, very long chain aldehydes, very long chain alkanes, very long chain ketones, or very long chain fatty ester waxes from C₁ substrates (such as methane or natural gas).

Background Description

Very long chain fatty acids are fatty acids with aliphatic tails having more than 24 carbons. They are composed of a nonpolar (lipophilic), saturated or unsaturated, hydrocarbon chain and a polar (hydrophilic) carboxyl group attached to the terminal carbon. Very long chain fatty acids may be incorporated into waxes or serve as precursors for other aliphatic hydrocarbons found in waxes, including alkanes, primary and secondary alcohols, ketones, aldehydes, and acyl-esters. Very long chain fatty acids and derivatives thereof are high value chemicals that may be used in the production of dietary supplements, food products, pharmaceutical formulations, lubricants, detergents, surfactants, cosmetics, nylon, coatings, adhesives, and biofuels.

The supply of very long fatty acids from natural sources and chemical synthesis is not sufficient for commercial needs. Obtaining very long fatty acids via natural sources or chemical synthesis either require harsh production environments, expensive starting materials, use of limited environmental resources, or production of detrimental byproducts. Increasing efforts have been made to bioengineer production of very long chain fatty acids. Much work has focused on production in seed oil of transgenic plants. Recombinant microorganisms, such as E. coli and various yeasts, have also been used to convert biomass-derived feedstock to very long chain fatty acids. However, even with the use of relatively inexpensive cellulosic biomass as a feedstock, more than half the mass of a carbohydrate feedstock is comprised of oxygen, which represents a significant limitation in conversion efficiency. Very long chain fatty acids and their derivatives (such as very long chain fatty alcohols, very long chain fatty aldehydes, very long chain alkanes, very long chain wax esters, and very long chain ketones) have significantly lower oxygen content than the carbohydrate feedstock, which limits the theoretical yield since much of the carbohydrate oxygen must be eliminated as waste. Thus, the economics of production of very long chain fatty acids and their derivatives from a carbohydrate feedstock is prohibitively expensive.

In view of the limitations associated with carbohydrate-based fermentation methods for production of very long chain fatty acids and related compounds, there is a need in the art for alternative, cost-effective, and environmentally friendly methods for producing very long chain fatty acids. The present disclosure meets such needs, and further provides other related advantages.

### BRIEF SUMMARY

In certain aspects, the present disclosure is directed to a method for making a very long carbon chain compound by (A) culturing a non-natural C₁ metabolizing non-photosynthetic microorganism with a C₁ substrate feedstock, wherein the C₁ metabolizing non-photosynthetic microorganism comprises one or more recombinant nucleic acid molecules encoding the following enzymes: a β-ketoacyl-CoA synthase (KCS); a β-ketoacyl-CoA reductase (KCR); a β-hydroxy acyl-CoA dehydratase (HCD); an enoyl-CoA reductase (ECR); wherein the C₁ metabolizing non-photosynthetic microorganism converts the C₁ substrate into a very long carbon chain compound comprising a very long chain fatty acyl-CoA, a very long chain fatty aldehyde, a very long chain fatty alcohol, a very long chain fatty ester wax, a very long chain alkane, a very long chain ketone, or a combination thereof; and (B) recovering the very long carbon chain compound.

In a related aspect, the present disclosure provides a non-natural methanotroph, comprising one or more recombinant nucleic acid molecules encoding the following enzymes: a β-ketoacyl-CoA synthase (KCS); a β-ketoacyl-CoA reductase (KCR); a β-hydroxy acyl-CoA dehydratase (HCD); an enoyl-CoA reductase (ECR), wherein the methanotroph is capable of converting a C₁ substrate into a very long carbon chain compound comprising a very long chain fatty acyl-CoA, a very long chain fatty aldehyde, a very long chain fatty alcohol, a very long chain fatty ester wax, a very long chain alkane, a very long chain ketone, or a combination thereof. In certain embodiments, there are provided non-natural methanotrophs containing a recombinant nucleic acid molecule encoding a fatty acyl-CoA reductase, wherein the methanotroph is capable of converting a C₁ substrate into a very long chain fatty aldehyde.

In certain embodiments, there are provided non-natural methanotrophs containing a recombinant nucleic acid molecule encoding a heterologous fatty alcohol forming acyl-CoA reductase, or a recombinant nucleic acid molecule encoding a heterologous fatty acyl-CoA reductase, and a recombinant nucleic acid molecule encoding a heterologous aldehyde reductase, wherein the methanotroph is capable of converting a C₁ substrate into a very long chain fatty alcohol.

In further embodiments, provided are non-natural methanotrophs containing a recombinant nucleic acid molecule encoding a heterologous fatty alcohol forming acyl-CoA reductase and a recombinant nucleic acid molecule encoding a heterologous ester synthase, wherein the methanotroph is capable of converting a C₁ substrate into a very long chain fatty ester wax.

In certain embodiments, there are provided non-natural methanotrophs containing a recombinant nucleic acid molecule encoding a heterologous fatty acyl-CoA reductase, and a recombinant nucleic acid molecule encoding a heterologous aldehyde decarbonylase, wherein the methanotroph is capable of converting a C₁ substrate into a very long chain alkane.

In further embodiments, there are provided non-natural methanotrophs containing a recombinant nucleic acid molecule encoding a heterologous fatty acyl-CoA reductase, a recombinant nucleic acid molecule encoding a heterologous aldehyde decarbonylase, and a recombinant nucleic acid molecule encoding a heterologous alkane hydroxylase, wherein the methanotroph is capable of converting a C₁ substrate into a very long chain ketone.

In another aspect, the present disclosure provides a C₁ metabolizing microorganism biomass comprising a very long chain carbon compound composition, wherein the very long carbon chain compound containing biomass or a very long carbon chain compound composition therefrom has a δ¹³C of about −35‰ to about −50‰, −45‰ to about −35‰, or about −50‰ to about −40‰, or about −45‰ to about −65‰, or about −60‰ to about −70‰, or about −30‰ to about −70‰. In certain embodiments, a very long carbon chain compound composition comprises very long chain fatty acyl-CoA, very long chain fatty aldehyde, very long chain fatty alcohol, very long chain fatty ester wax, very long chain alkane, very long chain ketone, or any combination thereof. In still further embodiments, a very long carbon chain compound composition comprises C₂₅-C₅₀ very long chain fatty acyl-CoA, C₂₅-C₅₀ very long chain fatty aldehyde, C₂₅-C₅₀ very long chain fatty alcohol, C₂₅-C₅₀ very long chain fatty ester wax, C₂₅-C₅₀ very long chain alkane, or C₂₅-C₅₀ very long chain ketone. In yet further embodiments, a very long carbon chain compound composition comprises a majority (more than 50% w/w) of very long carbon chain compounds having carbon chain lengths ranging from C₂₅-C₅₀ or a majority of very long carbon chain compounds having carbon chain lengths of greater than C₂₄, or a very long carbon chain compound containing composition wherein at least 70% of the total very long carbon chain compound comprises C₂₅-C₅₀ very long carbon chain compound.

### BRIEF DESCRIPTION OF THE DRAWINGS

FIG. 1 shows an overview of a fatty acid elongation pathway for very long chain acyl CoA production.

FIG. 2 shows an overview of very long chain fatty primary alcohol production.

FIG. 3 shows an overview of very long chain fatty ester wax production.

FIG. 4 shows an overview of very long chain alkane production and very long chain ketone production.

FIG. 5 shows an overview of an acyl-CoA dependent FAR Pathway for fatty alcohol production.

FIG. 6 shows an overview of an acyl-CoA independent FAR pathway for fatty alcohol production.

FIG. 7 shows an overview of an acyl-CoA independent CAR pathway for fatty alcohol production.

FIG. 8 shows an overview of a ω-hydroxy fatty acid production pathway.

FIG. 9 shows an overview of a dicarboxylic acid production pathway.

FIG. 10 shows an overview of an acyl-CoA dependent FAR pathway for fatty ester production.

FIG. 11 shows a schematic of the δ¹³C distribution of various carbon sources.

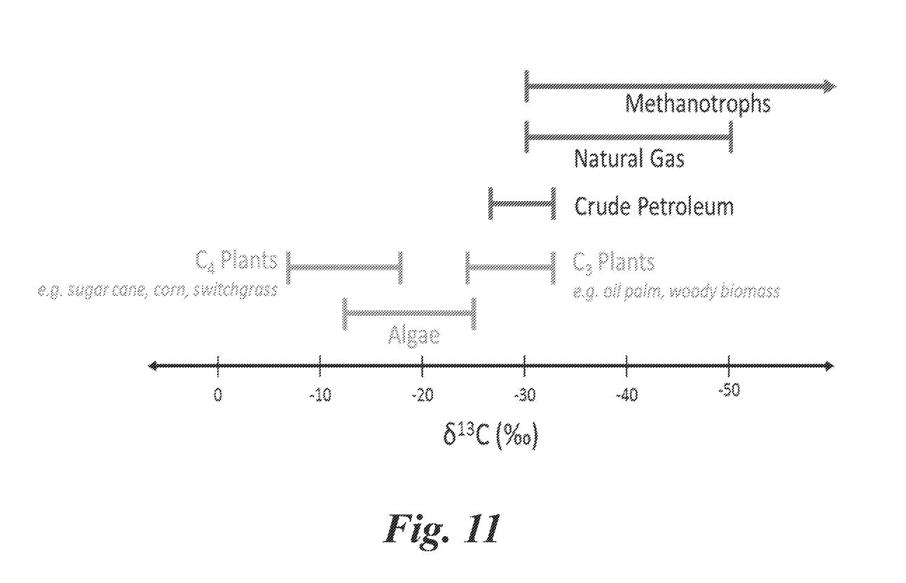

### DETAILED DESCRIPTION

The instant disclosure provides compositions and methods for generating very long chain carbon compounds. For example, recombinant C₁ metabolizing microorganisms are cultured with a C₁ substrate feedstock (e.g., methane) to generate greater than C₂₄ fatty acyl-CoA, fatty aldehyde, fatty alcohol, fatty ester wax, alkane, ketone, or any combination thereof. This new approach allows for the use of methylotroph or methanotroph bacteria as a new host system to generate very long chain fatty acid derivatives for use in producing, for example, dietary supplements, food products, pharmaceutical formulations, lubricants, detergents, surfactants, cosmetics, nylon, coatings, adhesives, or biofuels.

By way of background, methane from a variety of sources, including natural gas, represents an abundant domestic resource. As noted above, carbohydrate-based feedstocks contain more than half of their mass in oxygen, which is a significant limitation in conversion efficiency as very long chain fatty acids have significantly lower oxygen content than these feedstocks. A solution to address the limitations of current systems is to utilize methane or natural gas as a feedstock for conversion. Methane from natural gas is cheap and abundant, and importantly contains no oxygen, which allows for significant improvements in theoretical conversion efficiency. Furthermore, C₁ carbon sources are cheap and abundant compared to carbohydrate feedstock, which also contributes to improved economics of very long chain fatty acid production.

Very long chain fatty acid production is an important pathway in many different organisms as it is required for diverse physiological functions, such as skin barrier formation, retinal functions, resolution of inflammation, maintenance of myelin, sperm development and maturation, liver homeostasis, high membrane curvature in the nuclear pore, synthesis of GPI lipid anchor, and storage of triacylglycerols in plant seeds. Very long chain fatty acids are also components of plant cuticular waxes and membrane sphingolipids. Fatty acids are elongated in the form of acyl-CoA, in which fatty acids are linked to coenzyme A via thioester bonds. In the present disclosure, metabolic engineering techniques are applied to provide a fatty acid elongation pathway (e.g., one or more of a β-ketoacyl-CoA synthase, a β-ketoacyl-CoA reductase, a β-hydroxyacyl-CoA dehydratase, and an enoyl-CoA reductase) to allow production of very long chain fatty acyl-CoA from a fatty acyl-CoA substrate (e.g., C₁₆ or C₁₈ fatty acyl-CoA). In additional embodiments, a very long chain fatty acyl-CoA is further modified to produce a very long chain fatty aldehyde, a very long chain alkane, a very long chain fatty secondary alcohol, a very long chain ketone, or any combination thereof by introduction of various enzymes of an alkane forming pathway. In other embodiments, a very long chain fatty acyl-CoA is further modified to produce a very long chain aldehyde, very long chain fatty primary alcohol, very long chain wax ester, or any combination thereof by introduction of various enzymes of an alcohol forming pathway.

In one aspect, the present disclosure provides a method for producing a very long carbon chain compound, comprising culturing a non-natural C₁ metabolizing non-photosynthetic microorganism in the presence of a C₁ substrate feedstock, wherein the C₁ metabolizing non-photosynthetic microorganism comprises one or more recombinant nucleic acid molecules encoding the following enzymes: a β-ketoacyl-CoA synthase, a β-ketoacyl-CoA reductase, a β-hydroxyacyl-CoA dehydratase, and an enoyl-CoA reductase, wherein the C₁ metabolizing non-photosynthetic microorganism converts the C₁ substrate into a very long carbon chain compound; and recovering the very long carbon chain compound. In another aspect, this disclosure provides a non-natural methanotroph that includes one or more recombinant nucleic acid molecules encoding the following enzymes: a β-ketoacyl-CoA synthase, a β-ketoacyl-CoA reductase, a β-hydroxyacyl-CoA dehydratase, and an enoyl-CoA reductase, wherein the methanotroph is capable of converting a C₁ substrate into a very long carbon chain compound.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “recombinant” or “non-natural” refers to an organism, microorganism, cell, nucleic acid molecule, or vector that includes at least one genetic alternation or has been modified by the introduction of an exogenous nucleic acid, or refers to a cell that has been altered such that the expression of an endogenous nucleic acid molecule or gene can be controlled, where such alterations or modifications are introduced by genetic engineering. Genetic alterations include, for example, modifications introducing expressible nucleic acid molecules encoding proteins or enzymes, other nucleic acid additions, nucleic acid deletions, nucleic acid substitutions, or other functional disruption of the cell's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Exemplary proteins or enzymes include proteins or enzymes (i.e., components) within a very long chain fatty acid elongation pathway (e.g., β-ketoacyl-CoA synthase, β-ketoacyl-CoA reductase, β-hydroxyacyl-CoA dehydratase, enoyl-CoA reductase, or a combination thereof). Genetic modifications to nucleic acid molecules encoding enzymes, or functional fragments thereof, can confer a biochemical reaction capability or a metabolic pathway capability to the recombinant cell that is altered from its naturally occurring state.

The following abbreviations of enzyme names are used herein: “fatty acid elongase” is referred to as “FAE”; “β-ketoacyl-CoA synthase” or “3-ketoacyl-CoA synthase” is referred to as “KCS”; “β-ketoacyl-CoA reductase” or “3-ketoacyl-CoA reductase” is referred to as “KCR”; “β-hydroxyacyl-CoA dehydratase” or “3-hydroxyacyl-CoA dehydratase” is referred to as “HCD”; “enoyl-CoA reductase” is referred to as “ECR”; “diacylglycerol O-acyltransferase” is referred to as “DGAT”; “fatty acyl reductase” or “fatty alcohol forming acyl-CoA reductase” is referred to as “FAR”; “acyl carrier protein” is referred to as “ACP”; “coenzyme A” is referred to as “CoA”; “thioesterase” is referred to as “TE”; “fatty acid synthase” or “fatty acid synthetase” is referred to as “FAS”; “fatty acyl-CoA reductase” is referred to as “FACR”; “fatty acyl-CoA synthase” or “fatty acyl-CoA synthetase” or “acyl-CoA synthase” or “acyl-CoA synthetase” are used interchangeably herein and are referred to as “FACS”; and “acetyl-CoA carboxylase” is referred to as “ACC”.

Malonyl-CoA as used herein refers to a coenzyme A derivative of malonic acid of the structure COOH—(CO)—S-CoA. Malonyl-CoA is formed by carboxylating acetyl-CoA using acetyl-CoA carboxylase (ACC) enzyme.

Fatty acid elongase (FAE), as used herein, refers to a heterotetramer enzyme complex consisting of four distinct enzymes that add C₂ moieties donated from malonyl-CoA to a fatty acyl-CoA substrate sequentially to produce very long chain fatty acids. Each repeated FAE catalyzed fatty acid elongation cycle includes four consecutive enzymatic reactions (condensation, reduction, dehydration, and reduction) catalyzed by β-ketoacyl-CoA synthase, β-ketoacyl-CoA reductase, β-hydroxyacyl-CoA dehydratase, and enoyl-CoA reductase, respectively, which elongate a fatty acyl-CoA chain by two carbon chain units.

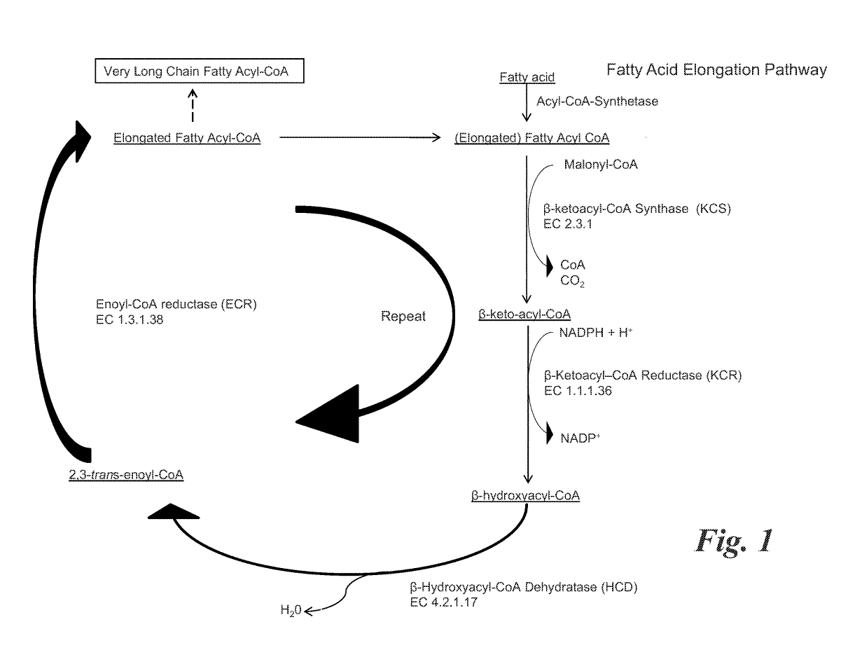

β-ketoacyl-CoA synthase (KCS), also known as 3-ketoacyl-CoA synthase or fatty acid elongase, as shown in FIG. 1 and used herein, refers to the rate limiting enzyme of the fatty acid elongation process, which condenses fatty acyl-CoA with malonyl-CoA to produce β-ketoacyl-CoA, also known as 3-ketoacyl-CoA. Acyl chain length substrate specificity of the very long chain fatty acid elongation cycle is thought to be determined by the KCS. A single KCS may catalyze condensation in a few consecutive elongation cycle. Various KCS enzymes may have overlapping ranges of acyl-CoA substrate chain lengths.

β-ketoacyl-CoA reductase (KCR) or 3-ketoacyl-CoA reductase as used herein refers to an enzyme that reduces β-ketoacyl-CoA to β-hydroxyacyl-CoA, also known as 3-hydroxyacyl-CoA (see FIG. 1). Nicotinamide adenine dinucleotide phosphate (NADPH) is used as a reducing agent in this reaction. A KCR may have broad compatibility for substrate chain length.

β-hydroxyacyl-CoA dehydratase (HCD) or 3-hydroxyacyl-CoA dehydratase as used herein refers to an enzyme that dehydrates β-hydroxyacyl-CoA into trans-enoyl-CoA, also known as 2,3-trans-enoyl-CoA (see FIG. 1). A HCD may have broad compatibility for substrate chain length.

Enoyl-CoA reductase (ECR) or 2,3-trans-enoyl-CoA reductase as used herein refers to an enzyme that reduces trans-enoyl-CoA to generate a fatty acyl-CoA having two additional carbon chain units than the original fatty acyl-CoA (see FIG. 1). NADPH is used as a reducing agent in this reaction. An ECR may have broad compatibility for substrate chain length.

Diacylglycerol O-acyltransferase (DGAT) or “O-acyltransferase,” as used herein, refers to an enzyme that forms triacylglycerols from diacylglycerol substrates and fatty acyl-CoAs

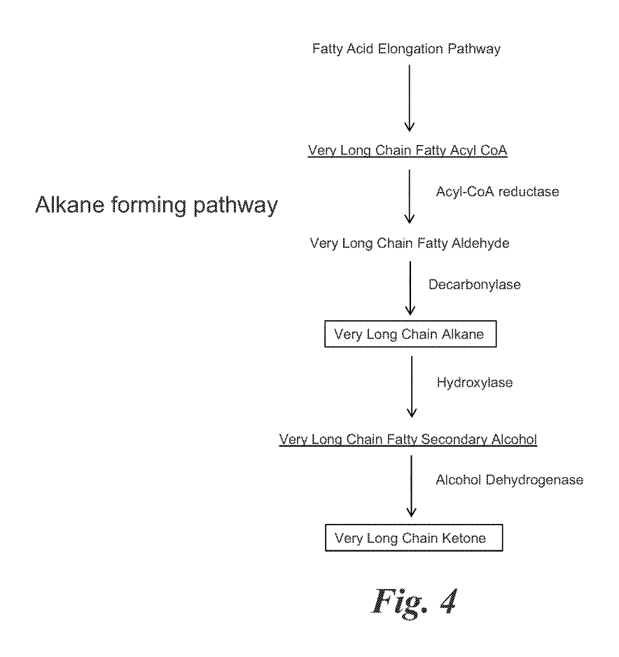

Aldehyde decarbonylase as used herein refers to an enzyme that decarbonylates a very long chain fatty aldehyde to generate a very long chain alkane, which has one less carbon chain unit than the very long chain fatty aldehyde substrate (see FIG. 4).

Alkane hydroxylase as used herein refers to an enzyme that catalyzes midchain hydroxylation of a very long chain alkane to generate a very long chain fatty secondary alcohol (see FIG. 4).

The phrase “fatty acid elongation pathway,” as used herein and shown in FIG. 1, refers to the elongation of a long chain fatty acid substrate (e.g., C₈ to C₂₄ fatty acyl-CoA) to a very long chain fatty acyl-CoA (greater than C₂₄) involving one or more elongation cycles that are catalyzed by KCS, KCR, HCD, and ECR. Each repeated elongation cycle extends the fatty acyl-CoA hydrocarbon chain by two carbons via a series of four reactions (condensation, reduction, dehydration, and reduction).

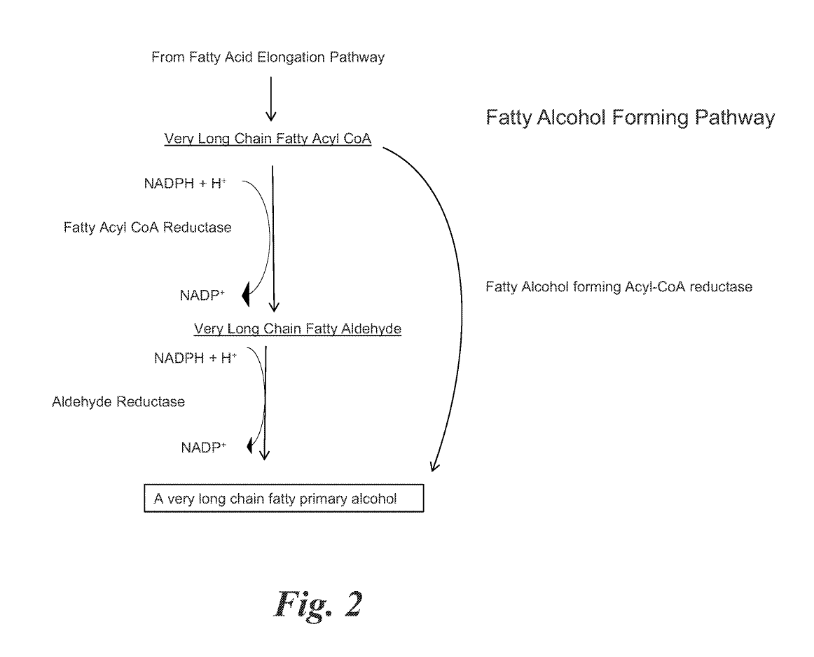

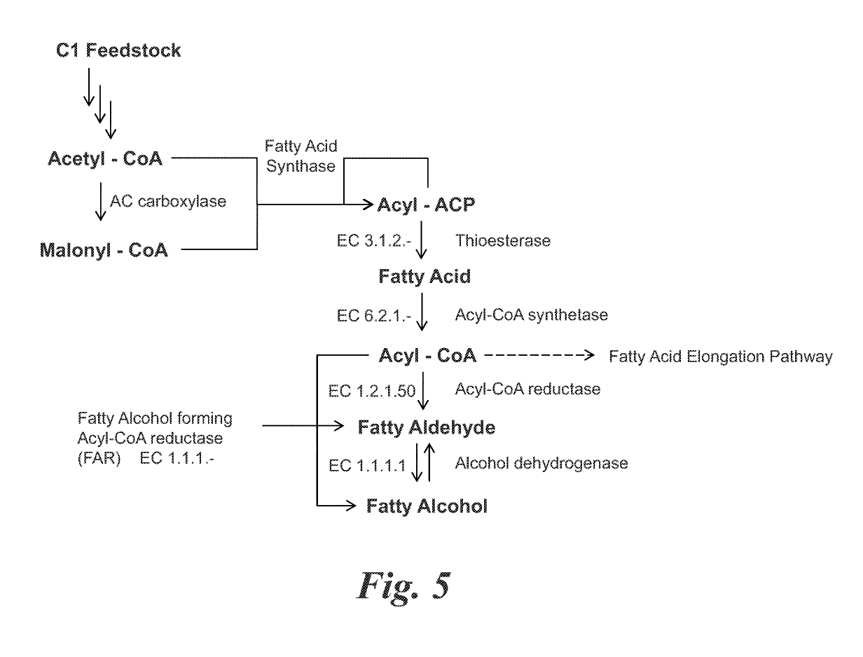

“Fatty Acyl Reductase” or “fatty alcohol forming acyl-CoA reductase” (FAR), as shown in FIGS. 1 and 2 and used herein, refers to an enzyme that catalyzes the reduction of a fatty acyl-CoA, a fatty acyl-ACP, or other fatty acyl thioester complex (each having a structure of R—(CO)—S—R₁, Formula I) to a fatty alcohol (structure R—OH, Formula II). For example, R—(CO)—S—R₁ (Formula I) is converted to R—OH (Formula II) and R₁—SH (Formula III) when two molecules of NADPH are oxidized to NADP⁺, wherein R is a C₈ to C₂₄ saturated, unsaturated, linear, branched or cyclic hydrocarbon, and R₁ represents CoA, ACP or other fatty acyl thioester substrate. FARs may also catalyze the reduction of a very long chain fatty acyl-CoA to a very long chain fatty alcohol. CoA is a non-protein acyl carrier group involved in the synthesis and oxidation of fatty acids. “ACP” is a polypeptide or protein subunit of FAS used in the synthesis of fatty acids. FARs are distinct from fatty acyl-CoA reductases (FACRs). FACRs reduce only fatty acyl-CoA or very long chain fatty acyl-CoA intermediates to fatty aldehydes or very long chain fatty aldehydes, respectively, and require an additional oxidoreductase enzyme to generate the corresponding fatty alcohol. Fatty aldehyde, as used herein (see FIG. 5), refers to a saturated or unsaturated aliphatic aldehyde, wherein R is as defined above. A very long chain fatty aldehyde is a fatty aldehyde, wherein R is at least a C₂₅ saturated, unsaturated, linear, branched or cyclic hydrocarbon.

The term “fatty acid” as used herein refers to a compound of structure R—COOH (Formula IV), wherein R is a C₈ to C₂₄ saturated, unsaturated, linear, branched or cyclic hydrocarbon and the carboxyl group is at position 1. Saturated or unsaturated fatty acids can be described as “Cx:y”, wherein “x” is an integer that represents the total number of carbon atoms and “y” is an integer that refers to the number of double bonds in the carbon chain. For example, a fatty acid referred to as C12:0 or 1-dodecanoic acid means the compound has 12 carbons and zero double bonds. The term “very long chain fatty acid” as used herein refers to a fatty acid wherein R is at least a C₂₅ saturated, unsaturated, linear, branched or cyclic hydrocarbon and the carboxyl group is at position 1.

The term “very long chain fatty wax ester” or “very long chain fatty ester wax” as used herein refers to an ester of a fatty acyl-CoA and a fatty alcohol wherein the number of carbon units is at least 25.

The term “very long chain alkane” as used herein refers to an at least C₂₅ linear or branched saturated hydrocarbon.

The term “very long chain ketone” as used herein refers to a compound of structure R—CO—R₁, wherein R and R₁ are independently saturated, unsaturated, linear, branched or cyclic hydrocarbons and the number of carbon units is at least 25.

The term “very long carbon chain compound” as used herein refers to a compound comprising a saturated, unsaturated, substantially linear carbon backbone having at least 25 carbon atoms. Very long carbon chain compounds include very long chain fatty acyl CoA, very long chain fatty aldehyde, very long chain fatty primary alcohol, very long chain fatty secondary alcohol, very long chain fatty ester wax, very long chain alkane, very long chain ketone, or any combination thereof.

The term “wax synthase” or “ester synthase” as used herein refers to an enzyme that conjugates a fatty alcohol to a fatty acyl-CoA via an ester linkage.

The term “aldehyde reductase” as used herein refers to an enzyme that reduces a very long chain fatty aldehyde to generate a very long chain fatty primary alcohol. NADPH is used as a reducing agent for this reaction. An aldehyde reductase may also refer to an alcohol dehydrogenase enzyme that may also be used to reduce a very long chain fatty aldehyde to generate a very long chain fatty primary alcohol.

The term “hydroxyl fatty acid” as used herein refers to a compound of structure OH—R—COOH (Formula V), wherein R is a C₈ to C₂₄ saturated, unsaturated, linear, branched or cyclic hydrocarbon. Omega hydroxy fatty acids (also known as ω-hydroxy acids) are a class of naturally occurring straight-chain aliphatic organic acids having a certain number of carbon atoms long with the carboxyl group at position 1 and a hydroxyl at position n. For example, exemplary C₁₆ ω-hydroxy fatty acids are 16-hydroxy palmitic acid (having 16 carbon atoms, with the carboxyl group at position 1 and the hydroxyl group at position 16) and 10,16-dihydroxy palmitic acid (having 16 carbon atoms, with the carboxyl group at position 1, a first hydroxyl group at position 10, and a second hydroxyl group at position 16).

The term “fatty alcohol” as used herein refers to an aliphatic alcohol of Formula II, wherein R is a C₈ to C₂₄ saturated, unsaturated, linear, branched or cyclic hydrocarbon. Saturated or unsaturated fatty alcohols can be described as “Cx:y-OH”, wherein “x” is an integer that represents the total number of carbon atoms in the fatty alcohol and “y” is an integer that refers to the number of double bonds in carbon chain. A “very long chain fatty alcohol” refers to a fatty alcohol wherein R is at least a C₂₅ saturated, unsaturated, linear, branched or cyclic hydrocarbon. A very long chain fatty primary alcohol refers to a very long chain alcohol which has the hydroxyl group connected to the primary carbon atom. A very long chain fatty secondary alcohol refers to a very long chain alcohol in which the carbon with the hydroxyl group attached is joined directly to two alkyl groups.

Unsaturated fatty acids or fatty alcohols can be referred to as “cisΔ^{z}” or “transΔ^{z}”, wherein “cis” and “trans” refer to the carbon chain configuration around the double bond and “z” indicates the number of the first carbon of the double bond, wherein the numbering begins with the carbon having the carboxylic acid of the fatty acid or the carbon bound to the —OH group of the fatty alcohol.

The term “fatty acyl-thioester” or “fatty acyl-thioester complex” refers to a compound of Formula I, wherein a fatty acyl moiety is covalently linked via a thioester linkage to a carrier moiety. Fatty acyl-thioesters are substrates for the FAR enzymes described herein.

The term “fatty acyl-CoA” refers to a compound of Formula I, wherein R₁ is Coenzyme A, and the term “fatty acyl-ACP” refers to a compound of Formula I, wherein R₁ is an acyl carrier protein ACP). The term “very long chain fatty acyl-CoA” refers to a fatty acyl-CoA wherein R is at least a C₂₅ saturated, unsaturated, linear, branched or cyclic hydrocarbon.

The phrase “acyl-CoA independent pathway” refers to the production of fatty alcohols by the direct enzymatic conversion of fatty acyl-ACP substrates to fatty alcohols and does not involve the use of free fatty acids or fatty acyl-CoA intermediates. This biosynthetic pathway differs from two types of fatty acyl-CoA dependent pathways one that converts fatty acyl-ACP directly to fatty acyl CoA via an acyl-transfer reaction, and a second that converts fatty acyl-ACP to fatty acyl-CoA via a free fatty acid intermediate (see FIG. 5). The acyl-CoA independent pathway has the advantage of bypassing the step of forming a fatty acyl-CoA substrate from free fatty acid, which requires the use of ATP. Therefore, the acyl-CoA independent pathway may use less energy than the acyl-CoA dependent pathway that utilizes a free fatty acid intermediate.

As used herein, “alcohol dehydrogenase” (ADH) refers to any enzyme capable of converting an alcohol into its corresponding aldehyde, ketone, or acid, and may also catalyze the reverse reaction. An alcohol dehydrogenase may have general specificity, capable of converting at least several alcohol substrates, or may have narrow specificity, accepting one, two or a few alcohol substrates. An alcohol dehydrogenase may be used to catalyze the oxidation of a very long chain secondary fatty alcohol to generate a very long chain ketone. An alcohol dehydrogenase may be used to catalyze the conversion of a very long chain fatty aldehyde to a very long chain fatty primary alcohol.

As used herein, “particulate methane monooxygenase” (pMMO) refers to a membrane-bound particulate enzyme that catalyzes the oxidation of methane to methanol in methanotrophic bacteria. The term pMMO means either the multi-component enzyme or the subunit comprising the enzyme's active site.

As used herein, “soluble methane monooxygenase” (sMMO) refers to an enzyme found in the soluble fraction of cell lysates (cytoplasm) that catalyzes the oxidation of methane to methanol in methanotrophic bacteria. The term sMMO means either the multi-component enzyme or the subunit comprising the enzyme's active site.

As used herein, “P450,” also known as “cytochrome P450” or “CYP,” refers to a group of enzymes with broad substrate specificity that catalyze the oxidation of organic compounds, including lipids, steroidal hormones, and xenobiotic substances. The P450 enzyme most commonly catalyzes a monooxgenase reaction by inserting an oxygen atom into the R-H bond of an organic substrate.

“Conversion” refers to the enzymatic conversion of a substrate to one or more corresponding products. “Percent conversion” refers to the percent of substrate that is reduced to one or more products within a period of time under specified conditions. Thus, the “enzymatic activity” or “activity” of a polypeptide enzyme can be expressed as “percent conversion” of a substrate to product.

As used herein, the term “host” refers to a microorganism (e.g., methanotroph) that is being genetically modified with very long chain fatty acid biosynthesis components (e.g., KCS, KCR, HCD, ECR, or any combination thereof) to convert a C₁ substrate feedstock into an at least a C₂₅ fatty acyl-CoA, fatty aldehyde, fatty alcohol, fatty ester wax, alkane, ketone or any combination thereof. A host cell may already possess other genetic modifications that confer desired properties unrelated to the very long chain fatty acid biosynthesis pathway disclosed herein. For example, a host cell may possess genetic modifications conferring high growth, tolerance of contaminants or particular culture conditions, ability to metabolize additional carbon substrates, or ability to synthesize desirable products or intermediates.

As used herein, the term “methanotroph,” “methanotrophic bacterium” or “methanotrophic bacteria” refers to a methylotrophic bacteria capable of utilizing C₁ substrates, such as methane or unconventional natural gas, as its primary or sole carbon and energy source. As used herein, “methanotrophic bacteria” include “obligate methanotrophic bacteria” that can only utilize C₁ substrates for carbon and energy sources and “facultative methanotrophic bacteria” that are naturally able to use multi-carbon substrates, such as acetate, pyruvate, succinate, malate, or ethanol, in addition to C₁ substrates as their sole carbon and energy source. Facultative methanotrophs include some species of Methylocella, Methylocystis, and Methylocapsa (e.g., Methylocella silvestris, Methylocella palustris, Methylocella tundrae, Methylocystis daltona SB2, Methylocystis bryophila, and Methylocapsa aurea KYG), and Methylobacterium organophilum (ATCC 27,886).

As used herein, the term “C₁ substrate” or “C₁ compound” refers to an organic compound lacking carbon to carbon bonds. C₁ substrates include syngas, natural gas, unconventional natural gas, methane, methanol, formaldehyde, formic acid (formate), carbon monoxide, carbon dioxide, methylated amines (e.g., methylamine, dimethylamine, trimethylamine, etc.), methylated thiols, methyl halogens (e.g., bromomethane, chloromethane, iodomethane, dichloromethane, etc.), and cyanide.

As used herein, “C₁ metabolizing microorganism” or “C₁ metabolizing non-photosynthetic microorganism” refers to any microorganism having the ability to use a C₁ substrate as a source of energy or as its primary source of energy or as its sole source of energy and biomass, and may or may not use other carbon substrates (such as sugars and complex carbohydrates) for energy and biomass. For example, a C₁ metabolizing microorganism may oxidize a C₁ substrate, such as methane, natural gas, or methanol. C₁ metabolizing microorganisms include bacteria (such as methanotrophs and methylotrophs). In certain embodiments, a C₁ metabolizing microorganism does not include a photosynthetic microorganism, such as algae. In certain embodiments, a C₁ metabolizing microorganism will be an “obligate C₁ metabolizing microorganism,” meaning its primary source of energy are C₁ substrates. In further embodiments, a C₁ metabolizing microorganism (e.g., methanotroph) will be cultured in the presence of a C₁ substrate feedstock (i.e., using the C₁ substrate as the primary or sole source of energy).

As used herein, the term “methylotroph” or “methylotrophic bacteria” refers to any bacteria capable of oxidizing organic compounds that do not contain carbon—carbon bonds. In certain embodiments, a methylotrophic bacterium may be a methanotroph. For example, “methanotrophic bacteria” refers to any methylotrophic bacteria that have the ability to oxidize methane as it primary source of carbon and energy. Exemplary methanotrophic bacteria include Methylomonas, Methylobacter, Methylococcus, Methylosinus, Methylocystis, Methylomicrobium, or Methanomonas. In certain other embodiments, the methylotrophic bacterium is an “obligate methylotrophic bacterium,” which refers to bacteria that are limited to the use of C₁ substrates for the generation of energy.

As used herein, the term “CO utilizing bacterium” refers to a bacterium that naturally possesses the ability to oxidize carbon monoxide (CO) as a source of carbon and energy. Carbon monoxide may be utilized from “synthesis gas” or “syngas”, a mixture of carbon monoxide and hydrogen produced by gasification of any organic feedstock, such as coal, coal oil, natural gas, biomass, and waste organic matter. CO utilizing bacterium does not include bacteria that must be genetically modified for growth on CO as its carbon source.

As used herein, “natural gas” refers to naturally occurring gas mixtures that have formed in porous reservoirs and can be accessed by conventional processes (e.g., drilling) and are primarily made up of methane, but may also have other components such as carbon dioxide, nitrogen or hydrogen sulfide.

As used herein, “unconventional natural gas” refers to a naturally occurring gas mixture created in formations with low permeability that must be accessed by unconventional methods, such as hydraulic fracturing, horizontal drilling or directional drilling. Exemplary unconventional natural gas deposits include tight gas sands formed in sandstone or carbonate, coal bed methane formed in coal deposits and adsorbed in coal particles, shale gas formed in fine-grained shale rock and adsorbed in clay particles or held within small pores or microfractures, methane hydrates that are a crystalline combination of natural gas and water formed at low temperature and high pressure in places such as under the oceans and permafrost.

As used herein, “syngas” refers to a mixture of carbon monoxide (CO) and hydrogen (H₂). Syngas may also include CO₂, methane, and other gases in smaller quantities relative to CO and H₂.

As used herein, “methane” refers to the simplest alkane compound with the chemical formula CH₄. Methane is a colorless and odorless gas at room temperature and pressure. Sources of methane include natural sources, such as natural gas fields, “unconventional natural gas” sources (such as shale gas or coal bed methane, wherein content will vary depending on the source), and biological sources where it is synthesized by, for example, methanogenic microorganisms, and industrial or laboratory synthesis. Methane includes pure methane, substantially purified compositions, such as “pipeline quality natural gas” or “dry natural gas”, which is 95-98% percent methane, and unpurified compositions, such as “wet natural gas”, wherein other hydrocarbons have not yet been removed and methane comprises more than 60% of the composition.

As used herein, “nucleic acid molecule,” also known as a polynucleotide, refers to a polymeric compound comprised of covalently linked subunits called nucleotides. Nucleic acid molecules include polyribonucleic acid (RNA), polydeoxyribonucleic acid (DNA), both of which may be single or double stranded. DNA includes cDNA, genomic DNA, synthetic DNA, semi-synthetic DNA, or the like.

As used herein, “transformation” refers to the transfer of a nucleic acid molecule (e.g., exogenous or heterologous nucleic acid molecule) into a host. The transformed host may carry the exogenous or heterologous nucleic acid molecule extra-chromosomally or the nucleic acid molecule may integrate into the chromosome. Integration into a host genome and self-replicating vectors generally result in genetically stable inheritance of the transformed nucleic acid molecule. Host cells containing the transformed nucleic acids are referred to as “recombinant” or “non-naturally occurring” or “genetically engineered” or “transformed” or “transgenic” cells (e.g., bacteria).

As used herein, the term “endogenous” or “native” refers to a gene, protein, compound or activity that is normally present in a host cell.

As used herein, “heterologous” nucleic acid molecule, construct or sequence refers to a nucleic acid molecule or portion of a nucleic acid molecule sequence that is not native to a host cell or is a nucleic acid molecule with an altered expression as compared to the native expression levels in similar conditions. For example, a heterologous control sequence (e.g., promoter, enhancer) may be used to regulate expression of a native gene or nucleic acid molecule in a way that is different from the way a native gene or nucleic acid molecule is normally expressed in nature or culture. In certain embodiments, heterologous nucleic acid molecules may not be endogenous to a host cell or host genome, but instead may have been added to a host cell by conjugation, transformation, transfection, electroporation, or the like, wherein the added molecule may integrate into the host genome or can exist as extra-chromosomal genetic material (e.g., as a plasmid or other self-replicating vector). In addition, “heterologous” can refer to an enzyme, protein or other activity that is different or altered from that found in a host cell, or is not native to a host cell but instead is encoded by a nucleic acid molecule introduced into the host cell. The term “homologous” or “homolog” refers to a molecule or activity found in or derived from a host cell, species or strain. For example, a heterologous nucleic acid molecule may be homologous to a native host cell gene, but may have an altered expression level or have a different sequence or both.

In certain embodiments, more than one heterologous nucleic acid molecules can be introduced into a host cell as separate nucleic acid molecules, as a polycistronic nucleic acid molecule, as a single nucleic acid molecule encoding a fusion protein, or any combination thereof, and still be considered as more than one heterologous nucleic acid. For example, as disclosed herein, a C₁ metabolizing microorganism can be modified to express two or more heterologous or exogenous nucleic acid molecules encoding desired very long chain fatty acid elongation pathway components (e.g., a β-ketoacyl-CoA synthase, a β-ketoacyl-CoA reductase, a β-hydroxyacyl-CoA dehydratase, and an enoyl-CoA reductase). When two or more exogenous nucleic acid molecules encoding very long chain fatty acid elongation pathway components are introduced into a host C₁ metabolizing microorganism, it is understood that the two more exogenous nucleic acid molecules can be introduced as a single nucleic acid molecule, for example, on a single vector, on separate vectors, can be integrated into the host chromosome at a single site or multiple sites, and still be considered two or more exogenous nucleic acid molecules. Thus, the number of referenced heterologous nucleic acid molecules or protein activities refers to the number of encoding nucleic acid molecules or the number of protein activities, not the number of separate nucleic acid molecules introduced into a host cell.

The term “chimeric” refers to any nucleic acid molecule or protein that is not endogenous and comprises sequences joined or linked together that are not normally found joined or linked together in nature. For example, a chimeric nucleic acid molecule may comprise regulatory sequences and coding sequences that are derived from different sources, or regulatory sequences and coding sequences that are derived from the same source but arranged in a manner different than that found in nature.

The “percent identity” between two or more nucleic acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp.71-77; Lewin, Genes IV, Oxford University Press, NY and Cell Press, Cambridge, Mass. (1990), p. 8).

“Inhibit” or “inhibited,” as used herein, refers to an alteration, reduction, down regulation or abrogation, directly or indirectly, in the expression of a target gene or in the activity of a target molecule (e.g., thioesterase, acyl-CoA synthetase, alcohol dehydrogenase) relative to a control, endogenous or reference molecule, wherein the alteration, reduction, down regulation or abrogation is statistically, biologically, industrially, or clinically significant.

As used herein, the term “derivative” refers to a modification of a compound by chemical or biological means, with or without an enzyme, which modified compound is structurally similar to a parent compound and (actually or theoretically) derivable from that parent compound. A derivative may have different chemical, biological or physical properties of the parent compound, such as being more hydrophilic or having altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). Other exemplary derivatizations include glycosylation, alkylation, acylation, acetylation, ubiqutination, esterification, and amidation. As used herein, “fatty acid derivatives” include intermediates and products of the fatty acid biosynthesis pathway found in cells, such as fatty acyl carrier proteins, activated fatty acids (e.g., acyl or CoA containing), fatty aldehydes, fatty alcohols, fatty ester wax, hydroxy fatty acids, dicarboxylic acids, branched fatty acids, or the like. As used herein, “very long chain fatty acid derivatives” include very long chain carbon compound intermediates and products of the very long chain fatty acid elongation pathway, alkane forming pathway, and alcohol forming pathway, such as very long chain fatty acids (e.g., acyl or CoA containing), very long chain fatty aldehydes, very long chain fatty alcohols, very long chain fatty ester waxes, very long chain alkanes, very long chain ketones, or the like.

The term “derivative” also refers to all solvates, for example, hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example, with inorganic acids such as hydrochloric acid, sulfuric acid or phosphoric acid, or with organic carboxylic acids and sulfonic acids such as acetic acid, citric acid, lactic acid, benzoic acid, maleic acid, fumaric acid, tartaric acid, methanesulfonic acid or p-toluenesulfonic acid. Compounds that simultaneously contain a basic group and an acidic group, for example, a carboxyl group in addition to basic nitrogen atoms, can be present as zwitterions. Salts can be obtained by customary methods known to those skilled in the art, for example, by combining a compound with an inorganic or organic acid or base in a solvent or diluent, or from other salts by cation exchange or anion exchange.

### Compositions and Methods for Making Very Long Carbon Chain Compounds

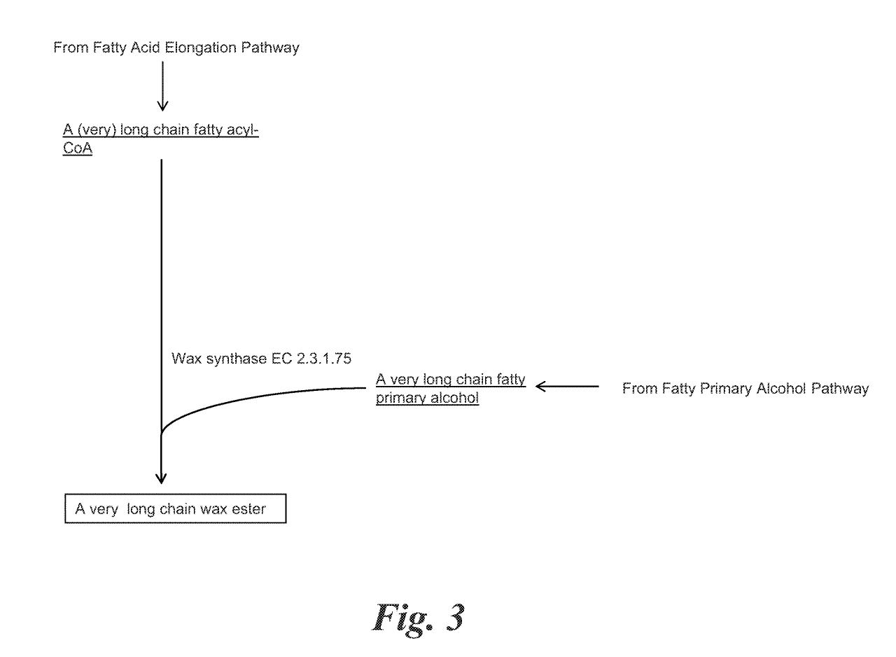

As described herein, very long carbon chain compound biosynthesis involves elongation of a fatty acid substrate (e.g., C₈-C₂₄ fatty acyl-CoA) by a fatty acid elongase (one or more of β-ketoacyl-CoA synthase, β-ketoacyl-CoA reductase, β-hydroxy acyl-CoA dehydratase, and enoyl-CoA reductase) through one or more cycles (see FIG. 1). Once elongated to the desired length, a very long chain fatty acid may be subsequently modified by either an alkane-forming (decarbonylation) pathway, which yields very long chain fatty aldehydes, very long chain alkanes, very long chain fatty secondary alcohols, or very long chain ketones (see FIG. 4), or an alcohol forming (acyl reduction) pathway, which yields very long chain fatty aldehydes, very long chain fatty primary alcohols, or very long chain fatty wax esters (see FIGS. 2-3). Fatty acid substrates for elongation to very long chain fatty acids may be synthesized naturally in a host C₁ metabolizing non-photosynthetic microorganism. Alternatively, a host C₁ metabolizing non-photosynthetic microorganism may be bioengineered to produce fatty acid substrates for elongation to very long chain fatty acids or to enhance endogenous production.

The C₁ metabolizing microorganisms used to produce very long carbon chain compounds can be recombinantly modified to include nucleic acid sequences that express or over-express polypeptides of interest. For example, a C₁ metabolizing microorganism can be modified to increase the production of acyl-CoA and reduce the catabolism of fatty acid derivatives and intermediates in the fatty acid biosynthetic pathway, such as acyl-CoA, or to reduce feedback inhibition at specific points in the fatty acid biosynthetic pathway. In addition to modifying the genes described herein, additional cellular resources can be diverted to over-produce fatty acids, for example, the lactate, succinate or acetate pathways can be attenuated, and acetyl-CoA carboxylase (acc) can be over-expressed. The modifications to a C₁ metabolizing microorganisms described herein can be through genomic alterations, addition of recombinant expression systems, or a combination thereof.

The very long carbon chain compound biosynthetic pathways are illustrated in FIGS. 1 to 4. Different steps in the pathway are catalyzed by different enzymes and each step is a potential place for over-expression of the gene to produce more enzyme and thus drive the production of more very long carbon chain compounds. Nucleic acid molecules encoding enzymes required for the pathway may also be recombinantly added to a C₁ metabolizing microorganism lacking such enzymes. Finally, steps that would compete with the pathway leading to production of very long carbon chain compounds can be attenuated or blocked in order to increase the production of the desired products.

In one aspect, provided herein are methods for making a very long carbon chain compound, the method comprising: (a) culturing a non-natural C₁ metabolizing non-photosynthetic microorganism with a C₁ substrate feedstock, wherein the C₁ metabolizing non-photosynthetic microorganism comprises one or more recombinant nucleic acid molecules encoding the following enzymes: (i) a β-ketoacyl-CoA synthase, (ii) a β-ketoacyl-CoA reductase, (iii) a β-hydroxy acyl-CoA dehydratase, and (iv) an enoyl-CoA reductase, wherein the C₁ metabolizing non-photosynthetic microorganism converts the C₁ substrate into a very long carbon chain compound. In certain embodiments, the C₁ metabolizing non-photosynthetic microorganism comprises two or more recombinant nucleic acid molecules encoding the following enzymes: (i) a β-ketoacyl-CoA ketoacyl-CoA synthase, (ii) a β-ketoacyl-CoA reductase, (iii) a β-hydroxy acyl-CoA dehydratase, and (iv) an enoyl-CoA reductase. In certain embodiments, the C₁ metabolizing non-photosynthetic microorganism comprises three or more recombinant nucleic acid molecules encoding the following enzymes: (i) a β-ketoacyl-CoA synthase, (ii) a β-ketoacyl-CoA reductase, (iii) a β-hydroxy acyl-CoA dehydratase, and (iv) an enoyl-CoA reductase. In certain embodiments, the C₁ metabolizing non-photosynthetic microorganism comprises recombinant nucleic acid molecules encoding all of the following enzymes: (i) a β-ketoacyl-CoA synthase, (ii) a β-ketoacyl-CoA reductase, (iii) a β-hydroxy acyl-CoA dehydratase, and (iv) an enoyl-CoA reductase. In certain embodiments, the very long carbon chain compound is a very long fatty acyl-CoA.

Fatty acid elongase is a heterotetrameric complex composed of four distinct enzymes that add C₂ moieties donated from malonyl-CoA to an acyl-CoA substrate to produce very long chain fatty acids. Each FAE catalyzed fatty acid elongation cycle consists of four consecutive enzymatic reactions (condensation, reduction, dehydration, and reduction) catalyzed by β-ketoacyl-CoA synthase, β-ketoacyl-CoA reductase, β-hydroxyacyl-CoA dehydratase, and enoyl-CoA reductase, respectively, which together elongate an acyl-CoA substrate chain by two carbon chain units. This elongation cycle has been described by Samuels et al. (Annu. Rev. Plant Biol. 59:683-707, 2008) and Kihara et al. (J. Biochem. 152:387-395, 2012).

β-ketoacyl-CoA synthase (KCS) is the rate limiting enzyme of the fatty acid elongation process. KCS catalyzes condensation of acyl-CoA with malonyl-CoA to produce β-ketoacyl-CoA, also known as 3-ketoacyl-CoA. Acyl chain length substrate specificity of the very long chain fatty acid elongation cycle is thought to be determined by the KCS. A single KCS may catalyze condensation in a few consecutive elongation cycle. Various KCSs may have overlapping ranges of acyl-CoA substrate chain lengths. For example, Arabidopsis KCS2/DAISY (Genbank Accession Identifier NM_100303.3) and KCS20 (Genbank Accession Identifier NM_123743.3) are involved in elongation of C₂₀ to C₂₂ very long chain fatty acids (Lee et al., 2009, Plant J. 60:462-75). Arabidopsis KCS9 (Genbank Accession Identifier NM_127184.2) is involved in elongation of C₂₂ to C₂₄ very long chain fatty acids (Kim et al., 2013, Plant Phsyiol. 162:567-80). Arabidopsis KCS1 (At1g01120) (Genbank Accession Identifier AF053345.1) has broad substrate specificity for saturated and mono-unsaturated C₁₆ to C₂₄ acyl-CoAs (Blacklock and Jawaorski, 2006, Biochem. Biophys. Res. Commun. 346:583-90). Mammals have seven KCS genes (ELOVL1-7), and each has a characteristic substrate specificity (Guillou et al., 2010, Prog. Lipid Res. 49:186-199; Ohno et al., 2010, Proc. Nat'l. Acad. Sci. USA 107:18439-18444). ELOVL6 elongates C16:0-CoA or shorter, saturatedacyl-CoAs. ELOVL3 and ELOVL7 elongate both saturated and unsaturated C₁₆-C₂₂ acyl-CoAs. ELOVL2 and ELOVL5 have strict specificity for polyunsaturated fatty acids and can elongate C₂₂-acyl-CoAs and C₁₈-CoAs, respectively, and both have overlapping specificity for C₂₀-acyl-CoAs. ELOVL1 elongates saturated C18:0-C26:0 and monounsaturated C20:1n-9 and C22:1n-9 acyl-CoAs. Arabidopsis CER6 (Genbank Accession Identifier NM_179530.1) has specificity for fatty acyl-CoA>C₂₂. Saccharomyces cerevisiae ELO1 (Genbank Accession Identifier NM_001181629) can elongate 14:0 to 16:0 fatty acids (Toke, 1996, J. Biol. Chem. 271:18413-18422). Saccharomyces cerevisiae ELO2 (Genbank Accession Identifier NM_001178748.1) can elongate fatty acids up to 24 carbons, and ELO3 (Genbank Accession Identifier NM_001182261.3) has broader substrate specificity and is essential for elongating C₂₄ to C₂₆ species (Oh et al., 1997, J. Biol. Chem. 272:17376-84). Genbank Accession Identifiers for other KCS genes include, for example, EU001741.1 (Gossypium hirsutum), EU001741.1 (Gossypium hirsutum), EU616538.1 (Solanum tuberosum), NM_001124636.1 (Oncorhynchus mykiss), JX436487.1 (Physcomitrella patens). In certain embodiments, a KCS gene is CER6, Elo1, Fen1/Elo2, Sur4/Elo3, KCS1, KCS2, KCS11, KCS20, KCS9, ELOVL1, ELOVL2, ELOVL3, ELOVL4, ELOVL5, ELOVL6, ELOVL7, or FDH.

β-ketoacyl-CoA reductase (KCR) also known as 3-ketoacyl-CoA reductase reduces β-ketoacyl-CoA to β-hydroxyacyl-CoA, also known as 3-hydroxyacyl-CoA. Nicotinamide adenine dinucleotide phosphate (NADPH) is used as a reducing agent in this reaction. KCRs are thought to have broad compatibility for substrate chain length. KCR genes include, for example, Saccharomyces cerevisiae YBR159w (Beaudoin et al., J. Biol. Chem., 2002, 277:11481-8), Arabidopsis AtKCR1 (At1g67730) (Beaudoin et al., 2009, Plant Physiol. 150:1174-1191), Zea mays L. GL8A and GL8B (Dietrich et al., 2005, Plant J. 42:844-61), Arabidopsis CER10 (Zhang et al., 2005, Plant Cell 17:1467-1481), and AYR1. In certain embodiments, a KCR gene is CER10, KAR, GL8A, GL8B, Ybr159w, AYR1, or At1g67730.

β-hydroxyacyl-CoA dehydratase (HCD) also known as 3-hydroxyacyl-CoA dehydratase dehydrates β-hydroxyacyl-CoA into trans-enoyl-CoA, also known as 2,3-trans-enoyl-CoA. HCDs are thought to have broad compatibility for substrate chain length. HCD genes include, for example, Arabidopsis PAS2 (Genbank Accession Identifier NM_001203348.1) (Bach et al. 2008, Proc. Natl. Acad. Sci. 105:14727-14731), Saccharomyces cerevisiae PHS1 (Genbank Accession Identifier NM_001181530.1), and mammalian isozymes HACD1 (Genbank Accession Identifier NM_014241.3, Homo sapiens), HACD2 (Genbank Accession Identifier NM_198402.3, Homo sapiens), HACD3 (Genbank Accession Identifier NM_016395.2, Homo sapiens), and HACD4 (Genbank Accession Identifier NM_001010915.3, Homo sapiens). In certain embodiments, an HCD gene is PHS1, PAS2, HACD1, HACD2, HACD3, HACD4, or PAS2-1.

Enoyl-CoA reductase (ECR) also known as 2,3-trans-enoyl-CoA reductase reduces trans-enoyl-CoA to generate a fatty acyl-CoA having two additional carbon chain units than the original fatty acyl-CoA substrate. NADPH is used a reducing agent in this reaction. ECRs are thought to have broad compatibility for substrate chain length. ECR genes include, for example, Arabidopsis CER10 (Genbank Accession Identifier NM_115394.3), Homo sapiens TER (Genbank Accession Identifier NM_138501.5), Saccharomyces cerevisiae TSC13 (Genbank Accession Identifier NM_01180074.1), Gossypium hirsutum GhECR1 (Genbank Accession Identifier EU001742.1), Gossypium hirsutum GhECR2 (Genbank Accession Identifier EU001743.1). In certain embodiments, an ECR is CER10, TER, TSC13, or GhECR1, GhECR2.

Exemplary KCS, KCR, HCD, and ECR genes from Nannochloropsis oculata, which are useful in the present disclosure, are also provided in PCT publication WO2012/052468.

The elongation cycle is repeated until a saturated fatty acid of the appropriate length is made. Odd chain length of very long chain fatty acyl-CoA may be generated by α-oxidation, which involves hydroxylation of the alpha carbon with an α-hydroxylase enzyme and decarboyxlation of an even chain length very long chain fatty acyl-CoA substrate.

To engineer a C₁ metabolizing microorganism for the production of a homogenous or mixed population of very long carbon chain compounds of particular carbon chain length(s), one or more KCS enzymes with a selected acyl chain length specificity can be expressed in the C₁ metabolizing microorganism. Additionally, one or more endogenous genes that produce very long chain fatty acids of undesirable length can be attenuated, inhibited, or functionally deleted.

Initial fatty acyl-CoA substrates for an elongation cycle may originate from endogenous fatty acid production in the C1 metabolizing microorganism. A fatty acyl-CoA pathway and enzymes involved are shown in FIG. 5. In certain embodiments, the initial fatty acyl-CoA substrate for an elongation cycle is a fatty acyl co-A with a carbon chain of about 8 to 24 carbon atoms, about 14 to 24 carbon atoms, about 10 to 20 carbon atoms, about 12 to 18 carbon atoms or about 16 to 18 carbon atoms.

In certain embodiments, the C₁ metabolizing non-photosynthetic microorganism further comprises a nucleic acid molecule that encodes a fatty alcohol forming acyl-CoA reductase (FAR) capable of forming a very long chain fatty alcohol, wherein the very long carbon chain compound is a very long chain fatty primary alcohol. One pathway for modification of a very long chain fatty acyl-CoA is the alcohol forming pathway (acyl reduction). The reduction of a very long chain fatty acid to its corresponding very long chain fatty primary alcohol goes through a very long chain fatty aldehyde intermediate and uses NADPH as a reducing agent for each reaction step. A FAR enzyme is capable of catalyzing both reactions without releasing a free aldehyde. A FAR gene includes, for example, Arabidopsis CER4 (Genbank Accession Identifier NM_119538.6) and Maqu_2220 (Genbank Accession Identifier YP_959486.1). The alcohol forming pathway for modifying very long chain fatty acyl-CoA has been described in Samuels et al., 2008, Annu Rev. Plant Biol. 59:683-707.

Alternatively, the reduction of a very long chain acyl-CoA to its corresponding very long chain fatty primary alcohol may be catalyzed by two independent enzymes. In certain embodiments, the C₁ metabolizing non-photosynthetic microorganism further comprises nucleic acid molecules that encode a fatty acyl-CoA reductase capable of forming a very long chain fatty aldehyde and an aldehyde reductase capable of forming a very long chain fatty alcohol, wherein the very long carbon chain compound is a very long chain fatty primary alcohol. A fatty acyl-CoA reductase gene includes, for example, Acinetobacter baylyi ACR1 (U77680.1), Synechococcus elongatus ACR (Lin et al., 2013, FEBS J. 280:4773-81) and Arabidopsis CER3 (Genbank Accession Identifier NM_125164.2). A very long chain fatty aldehyde may be reduced to a fatty alcohol by an aldehyde reductase or an NADPH-dependent alcohol dehydrogenase (e.g., YqhD). An aldehyde reductase gene includes, for example, YqhD.

In certain embodiments, the C₁ metabolizing non-photosynthetic microorganism further comprises nucleic acid molecule(s) encoding a fatty acyl-CoA reductase capable of forming a very long chain fatty aldehyde, wherein the very long carbon chain compound is a very long chain fatty aldehyde. A fatty acyl-CoA reductase gene includes, for example, ACR1, ACR, and Arabidopsis CER3 (Genbank Accession Identifier NM_125164.2).

In certain embodiments, the C₁ metabolizing non-photosynthetic microorganism comprises nucleic acid molecules encoding a fatty alcohol forming acyl-CoA reductase capable of forming a very long chain fatty alcohol and an ester synthase capable of forming a very long chain fatty ester wax, wherein the very long carbon chain compound is a very long chain fatty ester wax. A fatty primary alcohol (at least C₂₅), as described in detail herein, may also be conjugated by ester synthase with a fatty acyl-CoA (≦C₂₄), as described in detail herein, via an ester linkage to generate a very long chain fatty ester wax (>C₂₄). In other embodiments, a very long chain fatty ester wax may be generated by conjugating a very long chain fatty primary alcohol with a fatty acyl-CoA, a fatty primary alcohol with a very long chain fatty acyl-CoA, or a very long chain fatty primary alcohol with a very long chain fatty acyl-CoA via an ester synthase enzyme. An exemplary ester synthase gene includes, for example, Arabidopsis WSD1 (Li et al., 2008, Plant Phsyiol. 148:97-107; Genbank Accession Identifier NM_123089.2).

Very long chain fatty ester waxes are major components of waxes. A variety of natural and synthetic waxes are of industrial importance. In some embodiments, a C₁ metabolizing microorganism is modified so that it produces a very long chain fatty ester wax component of a natural or synthetic wax. Examples of natural waxes include beeswax, whale spermaceti, jojoba, carnauba, Chinese wax (insect wax), candelilla wax, and rice bran oil. The main components of beeswax are palmitate, palmitoleate, and oleate esters of very long chain (C₃₀-C₃₂) aliphatic alcohols. Sperm whale oil contains mostly fatty wax esters (65-95%) of cetyl palmitate (C₃₂) and cetyl myristate (C₃₀). Jojoba seed oil consists mainly of 18:1, 20:1 and 22:1 fatty acids linked to 20:1, 22:1 and 24:1 fatty alcohol, generating C₃₈-C₄₄ very long chain fatty ester waxes. Carnauba wax is composed mainly of very long chain fatty wax esters constituting C₁₆ to C₂₀ fatty acids linked to C₃₀ to C₃₄ alcohols, generating C₄₆ to C₅₄ wax esters. Major components of Chinese insect wax secreted by Coccu ceriferus are wax esters formed of chains with 46 up to 60 carbon atoms, the majority of alcohols and acids having 26 or 28 carbon atoms. Candelilla wax consists primarily of odd-numbered, saturated hydrocarbons (C₂₉ to C₃₃) along with esters of acids and alcohols with even-numbered carbon chains (C₂₈ to C₃₄). Rice bran oil contains esters of very long chain fatty acids (C₂₆ to C₃₀) and very long chain alcohols (C₂₆ to C₃₀).

A second pathway for modification of a very long chain fatty acyl-CoA is the alkane forming (decarbonylation) pathway. In certain embodiments, the C₁ metabolizing non-photosynthetic microorganism comprises nucleic acid molecule(s) encoding a fatty acyl-CoA reductase capable of forming a very long chain fatty aldehyde and an aldehyde decarbonylase capable of forming a very long chain alkane, wherein the very long carbon chain compound is a very long chain alkane. The first step is the reduction of a very long chain fatty acyl-CoA to its corresponding very long chain fatty aldehyde by acyl-CoA reductase. Removal of a carbonyl group by the aldehyde decarbonylase generates a very long chain alkane having one less carbon atom than its very long chain fatty acyl-CoA precursor. Alkane forming (decarbonylation) pathway has been described in Samuels et al., 2008, Annu Rev. Plant Biol. 59:683-707. A fatty acyl-CoA reductase gene includes, for example, ACR1 and Arabidopsis thaliana CER3 (Genbank Accession Identifier NM_125164.2). An aldehyde decarbonylase gene includes, for example, Arabidopsis thaliana CER1 (Genbank Accession Identifier D64155.1) and Arabidopsis CER22.

Further modification of the very long chain alkane by an alkane hydroxylase inserts a hydroxyl group mid-chain to generate a very long chain fatty secondary alcohol. The position of the hydroxyl group substitution depends upon the specificity of the hydroxylase. A hydroxylase gene includes, for example, Arabidopsis thaliana MAH1 (CYP96A15) (Greer et al., 2007, Plant Physiol. 145:653-667; Genbank Accession Identifier NM_001124037.1).

A second oxidation reaction of a very long chain fatty secondary alcohol, catalyzed by alcohol dehydrogenase, generates a very long chain ketone. In certain embodiments, the C₁ metabolizing non-photosynthetic microorganism comprises nucleic acid molecule(s) encoding a fatty acyl-CoA reductase capable of forming a very long chain fatty aldehyde, an aldehyde decarbonylase capable of forming a very long chain alkane, and an alkane hydroxylase capable of forming a very long chain fatty secondary alcohol, and an alcohol dehydrogenase capable of forming a very long chain ketone, wherein the very long carbon chain compound is a very long chain ketone. MAH1 (Genbank Accession Identifier NM_001124037.1) is also capable of performing this second oxidation reaction.

The enzymes described herein for generating fatty acyl-CoA substrates and fatty acid derivatives of 24 carbon units or less (e.g., acyl-CoA reductase, fatty alcohol forming acyl-CoA reductase, alcohol dehydrogenase) may also be used to further modify very long chain fatty acyl-CoA into derivatives thereof.

In certain embodiments, a very long carbon chain compound has a carbon chain length of about C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, or C₁₈₁-C₂₀₀. In alternative embodiments, a very long carbon chain compound is a C₂₅-C₄₀, C₂₅-C₅₀, C₂₅-C₇₅, C₂₅-C₁₀₀, C₂₅-C₁₂₅, C₂₅-C₁₅₀, C₂₅-C₁₇₅, or C₂₅-C₂₀₀ very long carbon chain compound.

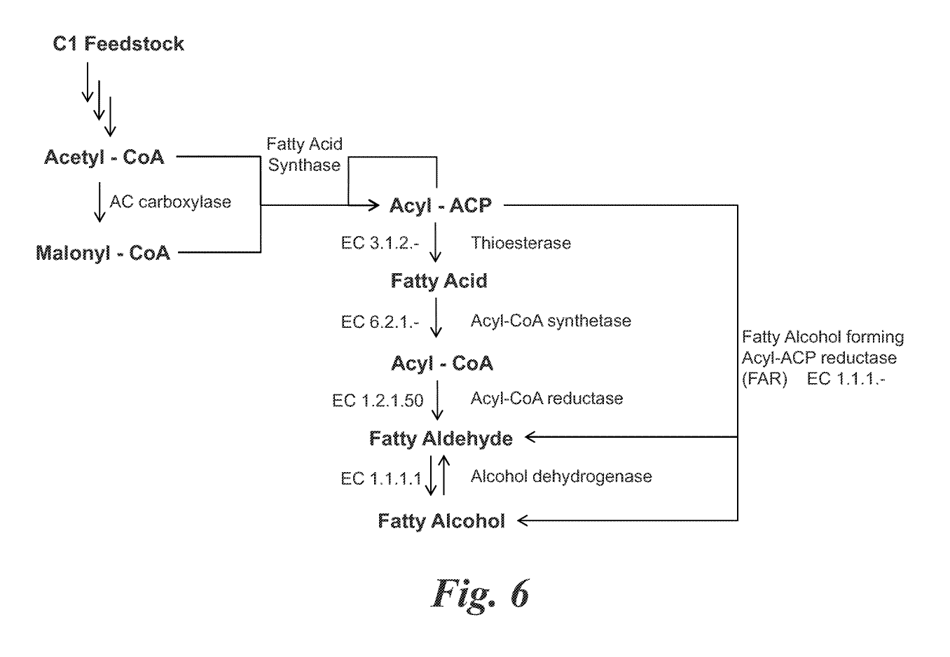

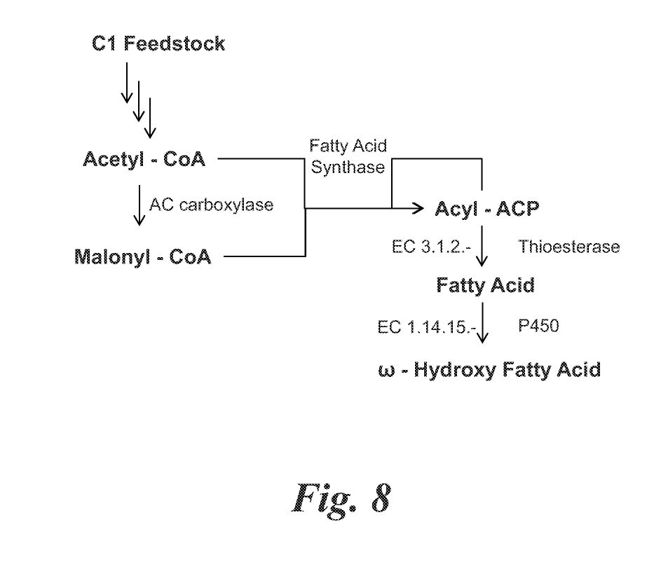

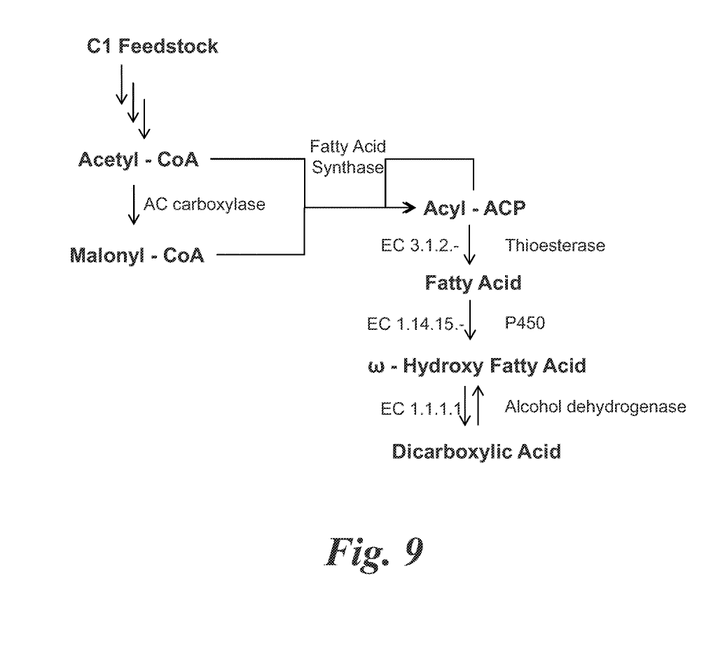

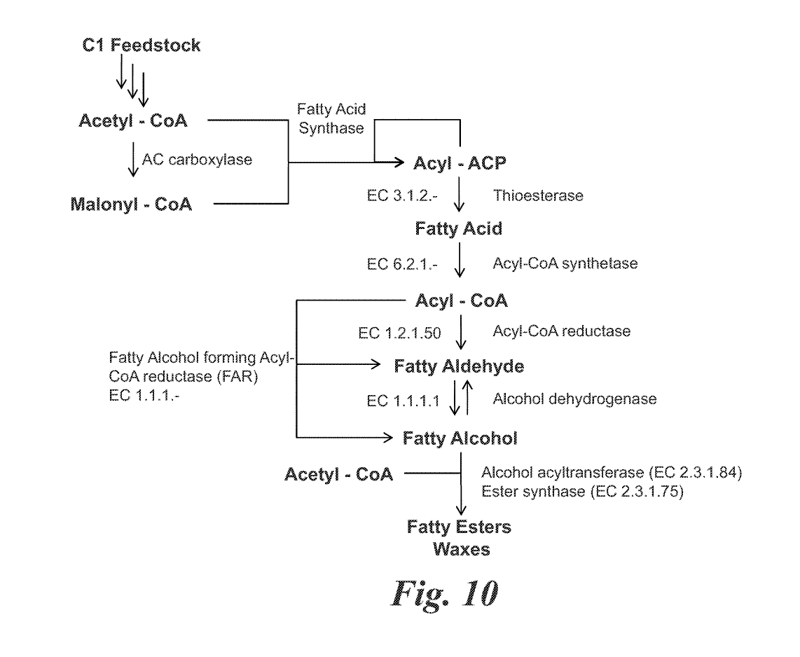

The fatty acyl-CoA substrates for elongation reactions to produce a very long chain fatty acyl-CoA may be produced endogenously by the C₁ metabolizing microorganisms. Alternatively, C₁ metabolizing microorganisms may be bioengineered to synthesize fatty acyl-CoA substrates for elongation. The fatty acid biosynthetic pathways involved are illustrated in FIGS. 5 to 10. Different steps in the pathway are catalyzed by different enzymes and each step is a potential place for over-expression of the gene to produce more enzyme and thus drive the production of more fatty acids and fatty acid derivatives. Nucleic acid molecules encoding enzymes required for the pathway may also be recombinantly added to a C₁ metabolizing microorganism lacking such enzymes. Finally, steps that would compete with the pathway leading to production of fatty acids and fatty acid derivatives can be attenuated or blocked in order to increase the production of the desired products.

Fatty acid synthases (FASs) are a group of enzymes that catalyze the initiation and elongation of acyl chains (Marrakchi et al., Biochemical Society 30:1050, 2002). The acyl carrier protein (ACP) along with the enzymes in the FAS pathway control the length, degree of saturation, and branching of the fatty acids produced. The steps in this pathway are catalyzed by enzymes of the fatty acid biosynthesis (fab) and acetyl-CoA carboxylase (acc) gene families. Depending upon the desired product, one or more of these genes can be attenuated, expressed or over-expressed (see FIGS. 5-10 for a depiction of the enzymatic activity of each enzyme and its enzyme classification number).

The fatty acid biosynthetic pathway in the production host uses the precursors acetyl-CoA and malonyl-CoA (see, e.g., FIG. 5). The steps in this pathway are catalyzed by enzymes of the fatty acid biosynthesis (fab) and acetyl-CoA carboxylase (acc) gene families. This pathway is described in Heath et al., Prog. Lipid Res. 40:467, 2001.

Acetyl-CoA is carboxylated by acetyl-CoA carboxylase (Acc, a multisubunit enzyme encoded by four separate genes, accABCD), to form malonyl-CoA. The malonate group is transferred to ACP by malonyl-CoA:ACP transacylase (FabD) to form malonyl-ACP. A condensation reaction then occurs, where malonyl-ACP merges with acetyl-CoA, resulting in β-ketoacyl-ACP. β-ketoacyl-ACP synthase III (FabH) initiates the FAS cycle, while β-ketoacyl-ACP synthase I (FabB) and β-ketoacyl-ACP synthase II (FabF) are involved in subsequent cycles.

Next, a cycle of steps is repeated until a saturated fatty acid of the appropriate length is made. First, the β-ketoacyl-ACP is reduced by NADPH to form β-hydroxyacyl-ACP. This step is catalyzed by β-ketoacyl-ACP reductase (FabG). β-hydroxyacyl-ACP is then dehydrated to form trans-2-enoyl-ACP. β-hydroxyacyl-ACP dehydratase/isomerase (FabA) or β-hydroxyacyl-ACP dehydratase (FabZ) catalyzes this step. NADPH-dependent trans-2-enoyl-ACP reductase I, II, or III (FabI, FabK, and FabL, respectively) reduces trans-2-enoyl-ACP to form acyl-ACP. Subsequent cycles are started by the condensation of malonyl-ACP with acyl-ACP by β-ketoacyl-ACP synthase I or β-ketoacyl-ACP synthase II (FabB and FabF, respectively).

C₁ metabolizing microorganisms as described herein may be engineered to overproduce acetyl-CoA and malonyl-CoA. Several different modifications can be made, either in combination or individually, to a C₁ metabolizing microorganism to obtain increased acetyl-CoA/malonyl-CoA/fatty acid, fatty acid derivative production, and very long carbon chain compound production.

For example, to increase acetyl-CoA production, one or more of the following genes could be expressed in a C₁ metabolizing microorganism: pdh, panK, aceEF (encoding the E1p dehydrogenase component and the E2p dihydrolipoamide acyltransferase component of the pyruvate and 2-oxoglutarate dehydrogenase complexes), fabH, fabD, fabG, acpP, or fabF. In other examples, additional DNA sequence encoding fatty-acyl-CoA reductases and aldehyde decarbonylases could be expressed in a C₁ metabolizing microorganism. It is well known in the art that a plasmid containing one or more of the aforementioned genes, all under the control of a constitutive, or otherwise controllable promoter, can be constructed. Exemplary GenBank accession numbers for these genes are pdh (BAB34380, AAC73227, AAC73226), panK (also known as coaA, AAC76952), aceEF (AAC73227, AAC73226), fabH (AAC74175), fabD (AAC74176), fabG (AAC74177), acpP (AAC74178), fabF (AAC74179).

Additionally, the expression levels of fadE, gpsA, ldhA, pflb, adhE, pta, poxB, ackA, or ackB can be reduced, inhibited or knocked-out in the engineered microorganism by transformation with conditionally replicative or non-replicative plasmids containing null or deletion mutations of the corresponding genes, or by substituting promoter or enhancer sequences. Exemplary GenBank accession numbers for these genes are fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting engineered C₁ metabolizing microorganisms will have increased acetyl-CoA production levels when grown in an appropriate environment, such as with a C₁ substrate feedstock.

Moreover, malonyl-CoA overproduction can be affected by engineering the C₁ metabolizing microorganisms as described herein with accABCD (e.g., accession number AAC73296, EC 6.4.1.2) included in the plasmid synthesized de novo. Fatty acid overproduction can be achieved by further including a nucleic acid molecule encoding lipase (e.g., Genbank Accession Nos. CAA89087, CAA98876) in the plasmid synthesized de novo.

As a result, in some examples, acetyl-CoA carboxylase is over-expressed to increase the intracellular concentration thereof by at least about 2-fold, preferably at least about 5-fold, or more preferably at least about 10-fold, relative to native expression levels.

In some embodiments, the plsB (e.g., Genbank Accession No. AAC77011) D311E mutation can be used to increase the amount of available acyl-CoA. In further embodiments, over-expression of a sfa gene (suppressor of FabA, e.g., Genbank Accession No. AAN79592) can be included in a C₁ metabolizing microorganism to increase production of monounsaturated fatty acids (Rock et al., J. Bacteriology 178:5382, 1996).

As described herein, acetyl-CoA and malonyl-CoA are processed in several steps to form acyl-ACP chains. The enzyme sn-glycerol-3-phosphate acyltransferase (PlsB) catalyzes the transfer of an acyl group from acyl-ACP or acyl-CoA to the sn-1 position of glycerol-3-phosphate. Thus, PlsB is a key regulatory enzyme in phospholipid synthesis, which is part of the fatty acid pathway. Inhibiting PlsB leads to an increase in the levels of long chain acyl-ACP, which feedback will inhibit early steps in the pathway (e.g., accABCD, fabH, and fabI). Uncoupling of this regulation, for example, by thioesterase overexpression leads to increased fatty acid production. The tes and fat gene families express thioesterase. FabI is also inhibited in vitro by long-chain acyl-CoA.

To engineer a C₁ metabolizing microorganism for the production of a homogeneous or mixed population of fatty acid derivatives, one or more endogenous genes can be attenuated, inhibited or functionally deleted and, as a result, one or more thioesterases can be expressed. For example, C₁₀ fatty acid derivatives can be produced by attenuating thioesterase C₁₈ (e.g., Genbank Accession Nos. AAC73596 and P0ADA1), which uses C_{18:1}-ACP, and at the same time expressing thioesterase C₁₀ (e.g., Genbank Accession No. Q39513), which uses C₁₀-ACP. This results in a relatively homogeneous population of fatty acid derivatives that have a carbon chain length of 10. In another example, C₁₄ fatty acid derivatives can be produced by attenuating endogenous thioesterases that produce non-C₁₄ fatty acids and expressing the thioesterase accession number Q39473 (which uses C₁₄-ACP). In yet another example, C₁₂ fatty acid derivatives can be produced by expressing thioesterases that use C₁₂-ACP (for example, Genbank Accession No. Q41635) and attenuating thioesterases that produce non-C₁₂ fatty acids. Thus, C₁ metabolizing microorganisms may be engineered to produce fatty acyl-CoA of preferred chain length(s) as substrates for subsequent elongation reactions initiated by KCS. Acetyl-CoA, malonyl-CoA, and fatty acid overproduction can be verified using methods known in the art, for example by using radioactive precursors, HPLC, and GC-MS subsequent to cell lysis. Non-limiting examples of thioesterases useful in the claimed methods and C₁ metabolizing microorganisms of this disclosure are listed in Table 1 of U.S. Pat. No. 8,283,143, which table is hereby incorporated by reference in its entirety.

Acyl-CoA synthase (ACS) esterifies free fatty acids to acyl-CoA by a two-step mechanism. The free fatty acid first is converted to an acyl-AMP intermediate (an adenylate) through the pyrophosphorolysis of ATP. The activated carbonyl carbon of the adenylate is then coupled to the thiol group of CoA, releasing AMP and the acyl-CoA final product. See Shockey et al., Plant. Physiol. 129:1710, 2002.

The E. coli ACS enzyme FadD and the fatty acid transport protein FadL are essential components of a fatty acid uptake system. FadL mediates transport of fatty acids into the bacterial cell, and FadD mediates formation of acyl-CoA esters. When no other carbon source is available, exogenous fatty acids are taken up by bacteria and converted to acyl-CoA esters, which bind to the transcription factor FadR and derepress the expression of the fad genes that encode proteins responsible for fatty acid transport (FadL), activation (FadD), and β-oxidation (FadA, FadB, FadE, and FadH). When alternative sources of carbon are available bacteria synthesize fatty acids as acyl-ACPs, which are used for phospholipid synthesis, but are not substrates for β-oxidation. Thus, acyl-CoA and acyl-ACP are both independent sources of fatty acids that will result in different end-products. See Caviglia et al., J. Biol. Chem. 279:1163, 2004.

C₁ metabolizing microorganisms can be engineered using nucleic acid molecules encoding known polypeptides to produce fatty acids of various lengths, which can then be converted to acyl-CoA and ultimately to very long carbon chain compounds. One method of making very long carbon chain compounds involves increasing the expression, or expressing more active forms, of one or more acyl-CoA synthase peptides (EC 6.2.1.-). A list of acyl-CoA synthases that can be expressed to produce acyl-CoA and fatty acid derivatives is shown in Table 2 of U.S. Pat. No. 8,283,143, which table is hereby incorporated by reference in its entirety. These acyl-CoA synthases can be used to improve any pathway that uses fatty-acyl-CoAs as substrates.

Acyl-CoA is reduced to a fatty aldehyde by NADH-dependent acyl-CoA reductase (e.g., Acr1). The fatty aldehyde is then reduced to a fatty alcohol by NADPH-dependent alcohol dehydrogenase (e.g., YqhD). Alternatively, fatty alcohol forming acyl-CoA reductase (FAR) catalyzes the reduction of an acyl-CoA into a fatty alcohol and CoASH. FAR uses NADH or NADPH as a cofactor in this four-electron reduction. Although the alcohol-generating FAR reactions proceed through an aldehyde intermediate, a free aldehyde is not released. Thus, alcohol-forming FARs are distinct from those enzymes that carry out two-electron reductions of acyl-CoA and yield free fatty aldehyde as a product. (See Cheng and Russell, J. Biol. Chem., 279:37789, 2004; Metz et al., Plant Physiol. 122:635, 2000).

C₁ metabolizing microorganisms can be engineered using known polypeptides to produce fatty alcohols from acyl-CoA. One method of making fatty alcohols involves increasing the expression of, or expressing more active forms of, fatty alcohol forming acyl-CoA reductases (encoded by a gene such as acr1 from FAR, EC 1.2.1.50/1.1.1) or acyl-CoA reductases (EC 1.2.1.50) and alcohol dehydrogenase (EC 1.1.1.1). Exemplary GenBank Accession Numbers are listed in FIG. 1 of U.S. Pat. No. 8,283,143, which figure is hereby incorporated by reference in its entirety.

Fatty alcohols can be described as hydrocarbon-based surfactants. For surfactant production, a C₁ metabolizing microorganism is modified so that it produces a surfactant from a C₁ substrate feedstock. Such a C₁ metabolizing microorganism includes a first exogenous nucleic acid molecule encoding a protein capable of converting a fatty acid to a fatty aldehyde and a second exogenous nucleic acid molecule encoding a protein capable of converting a fatty aldehyde to an alcohol. In some examples, a first exogenous nucleic acid molecule encodes a fatty acid reductase (FAR). In one embodiment, a second exogenous nucleic acid molecule encodes mammalian microsomal aldehyde reductase or long-chain aldehyde dehydrogenase. In a further example, first and second exogenous nucleic acid molecules are from Arthrobacter AK 19, Rhodotorula glutinins, Acinetobacter sp. M-1, or Candida lipolytica. In one embodiment, first and second heterologous nucleic acid molecules are from a multienzyme complex from Acinetobacter sp. M-1 or Candida lipolytica.

Additional sources of heterologous nucleic acid molecules encoding fatty acid to long chain alcohol converting proteins that can be used in surfactant production include Mortierella alpina (ATCC 32222), Cryptococcus curvatus, (also referred to as Apiotricum curvatum), Akanivorax jadensis (T9T=DSM 12718=ATCC 700854), Acinetobacter sp. HO1-N (ATCC 14987) and Rhodococcus opacus (PD630 DSMZ 44193).

In one example, a fatty acid derivative is a saturated or unsaturated surfactant product having a carbon chain length of about 8 to about 24 carbon atoms, about 8 to about 18 carbon atoms, about 8 to about 14 carbon atoms, about 10 to about 18 carbon atoms, or about 12 to about 16 carbon atoms. In another example, the surfactant product has a carbon chain length of about 10 to about 14 carbon atoms, or about 12 to about 14 carbon atoms.

Appropriate C₁ metabolizing microorganisms for producing surfactants can be either eukaryotic or prokaryotic microorganisms. C₁ metabolizing microorganisms that demonstrate an innate ability to synthesize high levels of surfactant precursors from C₁ feedstock in the form of fatty acid derivatives, such as methanogens engineered to express acetyl CoA carboxylase are used.

Production hosts can be engineered using known polypeptides to produce fatty esters of various lengths. One method of making fatty esters includes increasing the expression of, or expressing more active forms of, one or more alcohol O-acetyltransferase peptides (EC 2.3.1.84). These peptides catalyze the acetylation of an alcohol by converting an acetyl-CoA and an alcohol to a CoA and an ester. In some examples, the alcohol O-acetyltransferase peptides can be expressed in conjunction with selected thioesterase peptides, FAS peptides, and fatty alcohol forming peptides, thus allowing the carbon chain length, saturation, and degree of branching to be controlled. In some cases, a bkd operon can be coexpressed to enable branched fatty acid precursors to be produced.

As used herein, alcohol O-acetyltransferase peptides include peptides in enzyme classification number EC 2.3.1.84, as well as any other peptide capable of catalyzing the conversion of acetyl-CoA and an alcohol to form a CoA and an ester. Additionally, one of ordinary skill in the art will appreciate that alcohol O-acetyltransferase peptides will catalyze other reactions.

For example, some alcohol O-acetyltransferase peptides will accept other substrates in addition to fatty alcohols or acetyl-CoA thioester, such as other alcohols and other acyl-CoA thioesters. Such non-specific or divergent-specificity alcohol O-acetyltransferase peptides are, therefore, also included. Alcohol O-acetyltransferase peptide sequences are publicly available and exemplary GenBank Accession Numbers are listed in FIG. 1 of U.S. Pat. No. 8,283,143, which figure is hereby incorporated by reference in its entirety. Assays for characterizing the activity of particular alcohol O-acetyltransferase peptides are well known in the art. O-acyltransferases can be engineered to have new activities and specificities for the donor acyl group or acceptor alcohol moiety. Engineered enzymes can be generated through well-documented rational and evolutionary approaches.

Fatty esters are synthesized by acyl-CoA:fatty alcohol acyltransferase (e.g., ester synthase), which conjugate a long chain fatty alcohol to a fatty acyl-CoA via an ester linkage. Ester synthases and encoding genes are known from the jojoba plant and the bacterium Acinetobacter sp. ADP1 (formerly Acinetobacter calcoaceticus ADP1). The bacterial ester synthase is a bifunctional enzyme, exhibiting ester synthase activity and the ability to form triacylglycerols from diacylglycerol substrates and fatty acyl-CoAs (acyl-CoA:diglycerol acyltransferase (DGAT) activity). The gene wax/dgat encodes both ester synthase and DGAT. See Cheng et al., J. Biol. Chem. 279:37798, 2004; Kalscheuer and Steinbuchel, J. Biol. Chem. 278:8075, 2003. Ester synthases may also be used to produce certain fatty esters.

The production of fatty esters, including waxes, from acyl-CoA and alcohols, can be engineered using known polypeptides. One method of making fatty esters includes increasing the expression of, or expressing more active forms of, one or more ester synthases (EC 2.3.1.20, 2.3.1.75). Ester synthase peptide sequences are publicly available and exemplary GenBank Accession Numbers are listed in FIG. 1 of U.S. Pat. No. 8,283,143, which figure is hereby incorporated by reference in its entirety. Methods to identify ester synthase activity are provided in U.S. Pat. No. 7,118,896.

In particular examples, if a desired product is a fatty acid ester wax, a C₁ metabolizing microorganism is modified so that it produces an ester. Such a C₁ metabolizing microorganism includes an exogenous nucleic acid molecule encoding an ester synthase that is expressed so as to confer upon a C₁ metabolizing microorganism the ability to synthesize a saturated, unsaturated, or branched fatty ester from a C₁ substrate feedstock. In some embodiments, a C₁ metabolizing microorganism can also express nucleic acid molecules encoding the following exemplary proteins: fatty acid elongases, acyl-CoA reductases, acyltransferases, ester synthases, fatty acyl transferases, diacylglycerol acyltransferases, acyl-coA wax alcohol acyltransferases, or any combination thereof. In an alternate embodiment, C₁ metabolizing microorganisms comprises a nucleic acid molecule encoding a bifunctional ester synthase/acyl-CoA:diacylglycerol acyltransferase. For example, the bifunctional ester synthase/acyl-CoA:diacylglycerol acyltransferase can be selected from the multienzyme complexes from Simmondsia chinensis, Acinetobacter sp. ADP1 (formerly Acinetobacter calcoaceticus ADP1), Alcanivorax borkumensis, Pseudomonas aeruginosa, Fundibacter jadensis, Arabidopsis thaliana, or Alcaligenes eutrophus (later renamed Ralstonia eutropha). In one embodiment, fatty acid elongases, acyl-CoA reductases or wax synthases are from a multienzyme complex from Ralstonia eutropha or other organisms known in the literature to produce esters, such as wax or fatty esters.

Additional sources of heterologous nucleic acid molecules encoding ester synthesis proteins useful in fatty ester production include Mortierella alpina (e.g., ATCC 32222), Cryptococcus curvatus (also referred to as Apiotricum curvatum), Alcanivorax jadensis (for example, T9T=DSM 12718=ATCC 700854), Acinetobacter sp. HO1-N (e.g., ATCC 14987), and Rhodococcus opacus (e.g., PD630, DSMZ 44193). In one example, the ester synthase from Acinetobacter sp. ADP1 at locus AA017391 (described in Kalscheuer and Steinbuchel, J. Biol. Chem. 278:8075, 2003) is used. In another example, an ester synthase from Simmondsia chinensis at locus AAD38041 is used.

Optionally, an ester exporter such as a member of the FATP family can be used to facilitate the release of esters into the extracellular environment. A non-limiting example of an ester exporter that can be used is fatty acid (long chain) transport protein CG7400-PA, isoform A, from Drosophila melanogaster, at locus NP_524723.

Transport proteins export fatty acid derivatives out of a C₁ metabolizing microorganism. Many transport and efflux proteins serve to excrete a large variety of compounds, and can naturally be modified to be selective for particular types of fatty acid derivatives. Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, Rhodococcus erythropolis. Exemplary ABC transport proteins which could be used are CER5, AtMRP5, AmiS2, or AtPGP1. In a preferred embodiment, an ABC transport protein is CER5 (e.g., AY734542). Vectors containing genes that express suitable transport proteins can be inserted into the protein production host to increase the release of fatty acid derivatives.

C₁ metabolizing microorganisms can also be chosen for their endogenous ability to release fatty acid derivatives. The efficiency of product production and release into the fermentation broth can be expressed as a ratio of intracellular product to extracellular product. In some examples, the ratio can be about 5:1, 4:1, 3:1, 2:1, 1:1, 1:2, 1:3, 1:4, or 1:5.

Fatty acid derivatives with particular branch points, levels of saturation, carbon chain length, and ester characteristics can be produced as desired. C₁ metabolizing microorganisms that naturally produce particular derivatives can be chosen as the initial host cell. Alternatively, genes that express enzymes that will produce particular fatty acid derivatives can be inserted into a C₁ metabolizing microorganism as described herein.

In some examples, the expression of exogenous FAS genes originating from different species or engineered variants can be introduced into a C₁ metabolizing microorganism to allow for the biosynthesis of fatty acids that are structurally different (in length, branching, degree of unsaturation, etc.) from those of the native host cell. These heterologous gene products can also be chosen or engineered to be unaffected by the natural regulatory mechanisms in the host cell, and therefore allow for control of the production of the desired commercial product. For example, FAS enzymes from Bacillus subtilis, Saccharomyces cerevisiae, Streptomyces spp., Ralstonia, Rhodococcus, Corynebacteria, Brevibacteria, Mycobacteria, oleaginous yeast, or the like can be expressed in a C₁ metabolizing microorganism of this disclosure. The expression of such exogenous enzymes will alter the structure of the fatty acid produced and ultimately the fatty acid derivative.

When a C₁ metabolizing microorganism is engineered to produce a fatty acid with a specific level of unsaturation, branching, or carbon chain length, the resulting engineered fatty acid can be used in the production of fatty acid derivatives. Fatty acid derivatives generated from such C₁ metabolizing microorganisms can display the characteristics of the engineered fatty acid.

For example, a production host can be engineered to make branched, short chain fatty acids, which may then be used by the production host to produce branched, short chain fatty alcohols. Similarly, a hydrocarbon can be produced by engineering a production host to produce a fatty acid having a defined level of branching, unsaturation, or carbon chain length; thus, producing a homogeneous hydrocarbon population. Additional steps can be employed to improve the homogeneity of the resulting product. For example, when an unsaturated alcohol, fatty ester, or hydrocarbon is desired, a C₁ metabolizing microorganism can be engineered to produce low levels of saturated fatty acids and in addition can be modified to express an additional desaturase to lessen or reduce the production of a saturated product.

Fatty acids are a key intermediate in the production of fatty acid derivatives. Fatty acid derivatives can be produced to contain branch points, cyclic moieties, and combinations thereof, by using branched or cyclic fatty acids to make the fatty acid derivatives.

For example, C₁ metabolizing microorganisms may naturally produce straight chain fatty acids. To engineer C₁ metabolizing microorganisms to produce branched chain fatty acids, several genes that provide branched precursors (e.g., bkd operon) can be introduced into a C₁ metabolizing microorganism (e.g., methanogen) and expressed to allow initiation of fatty acid biosynthesis from branched precursors (e.g., fabH). The bkd, ilv, icm, and fab gene families may be expressed or over-expressed to produce branched chain fatty acid derivatives. Similarly, to produce cyclic fatty acids, genes that provide cyclic precursors can be introduced into the production host and expressed to allow initiation of fatty acid biosynthesis from cyclic precursors. The ans, chc, and plm gene families may be expressed or over-expressed to produce cyclic fatty acids. Non-limiting examples of genes in these gene families that may be used in the present methods and C₁ metabolizing microorganisms of this disclosure are listed in U.S. Pat. No. 8,283,143 (FIG. 1, which figure is herein incorporated by reference).

Additionally, the production host can be engineered to express genes encoding proteins for the elongation of branched fatty acids (e.g., ACP, FabF, etc.) or to delete or attenuate the corresponding genes that normally lead to straight chain fatty acids. In this regard, endogenous genes that would compete with the introduced genes (e.g., fabH, fabF) are deleted, inhibited or attenuated.

The branched acyl-CoA (e.g., 2-methyl-butyryl-CoA, isovaleryl-CoA, isobutyryl-CoA, etc.) are the precursors of branched fatty acids. In most microorganisms containing branched fatty acids, the branched fatty acids are synthesized in two steps from branched amino acids (e.g., isoleucine, leucine, and valine) (Kadena, Microbiol. Rev. 55:288, 1991). A C₁ metabolizing microorganism can be engineered to express or over-express one or more of the enzymes involved in these two steps to produce branched fatty acid derivatives, or to over-produce branched fatty acid derivatives. For example, a C₁ metabolizing microorganism may have an endogenous enzyme that can accomplish one step leading to branched fatty acid derivative; therefore, only genes encoding enzymes involved in the second step need to be introduced recombinantly.

The first step in forming branched fatty acid derivatives is the production of the corresponding α-keto acids by a branched-chain amino acid aminotransferase. C₁ metabolizing microorganisms, such as methanotrophs, may endogenously include genes encoding such enzymes or such genes may be recombinantly introduced. In some C₁ metabolizing microorganisms, a heterologous branched-chain amino acid aminotransferase may not be expressed. Hence, in certain embodiments, IlvE from E. coli or any other branched-chain amino acid aminotransferase (e.g., IlvE from Lactococcus lactis (GenBank accession AAF34406), IlvE from Pseudomonas putida (GenBank accession NP_745648), or IlvE from Streptomyces coelicolor (GenBank accession NP_629657)) can be introduced into C₁ metabolizing microorganisms of this disclosure. If the aminotransferase reaction is rate limiting in branched fatty acid biosynthesis in the chosen C₁ metabolizing microorganism, then an aminotransferase can be over-expressed.

The second step is the oxidative decarboxylation of the α-ketoacids to the corresponding branched-chain acyl-CoA. This reaction can be catalyzed by a branched-chain α-keto acid dehydrogenase complex (bkd; EC 1.2.4.4.) (Denoya et al., J. Bacteriol. 177:3504, 1995), which includes E1α/β (decarboxylase), E2 (dihydrolipoyl transacylase) and E3 (dihydrolipoyl dehydrogenase) subunits. These branched-chain α-keto acid dehydrogenase complexes are similar to pyruvate and α-ketoglutarate dehydrogenase complexes. Every microorganism that possesses branched fatty acids or grows on branched-chain amino acids can be used as a source to isolate bkd genes for expression in C₁ metabolizing microorganisms, such as methanotrophs. Furthermore, if the C₁ metabolizing microorganism has an E3 component as part of its pyruvate dehydrogenase complex (lpd, EC 1.8.1.4), then it may be sufficient to only express the E1α/β and E2 bkd genes.

In another example, isobutyryl-CoA can be made in a C₁ metabolizing microorganism, for example, in a methanotroph, through the coexpression of a crotonyl-CoA reductase (Ccr, EC 1.6.5.5, 1.1.1.1) and isobutyryl-CoA mutase (large subunit IcmA, EC 5.4.99.2; small subunit IcmB, EC 5.4.99.2) (Han and Reynolds, J. Bacteriol. 179:5157, 1997). Crotonyl-CoA is an intermediate in fatty acid biosynthesis in E. coli and other microorganisms.

In addition to expression of the bkd genes, the initiation of brFA biosynthesis utilizes β-ketoacyl-acyl-carrier-protein synthase III (FabH, EC 2.3.1.41) with specificity for branched chain acyl-CoAs (Li et al., J. Bacteriol. 187:3795, 2005). A fabH gene that is involved in fatty acid biosynthesis of any branched fatty acid-containing microorganism can be expressed in a C₁ metabolizing microorganism of this disclosure. The Bkd and FabH enzymes from production hosts that do not naturally make branched fatty acids or derivatives thereof may not support branched fatty acid production; therefore, Bkd and FabH can be expressed recombinantly. Vectors containing the bkd and fabH genes can be inserted into such a C₁ metabolizing microorganism. Similarly, the endogenous level of Bkd and FabH production may not be sufficient to produce branched fatty acid derivatives, so in certain embodiments they are over-expressed. Additionally, other components of the fatty acid biosynthesis pathway can be expressed or over-expressed, such as acyl carrier proteins (ACPs) and β-ketoacyl-acyl-carrier-protein synthase II (fabF, EC 2.3.1.41). In addition to expressing these genes, some genes in the endogenous fatty acid biosynthesis pathway may be attenuated in the C₁ metabolizing microorganisms of this disclosure. Genes encoding enzymes that would compete for substrate with the enzymes of the pathway that result in brFA production may be attenuated or inhibited to increase branched fatty acid derivative production.

As mentioned above, branched chain alcohols can be produced through the combination of expressing genes that support branched fatty acid synthesis and alcohol synthesis. For example, when an alcohol reductase, such as Acr1 from Acinetobacter baylyi ADP1, is coexpressed with a bkd operon, C₁ metabolizing microorganisms of this disclosure can synthesize isopentanol, isobutanol or 2-methyl butanol. Similarly, when Acr1 is coexpressed with ccrlicm genes, C₁ metabolizing microorganisms of this disclosure can synthesize isobutanol.

To convert a C₁ metabolizing microorganisms of this disclosure, such as a methanotroph, into an organism capable of synthesizing ω-cyclic fatty acids (cyFA), a gene that provides the cyclic precursor cyclohexylcarbonyl-CoA (CHC-CoA) (Cropp et al., Nature Biotech. 18:980, 2000) is introduced and expressed in the C₁ metabolizing microorganisms of this disclosure.

Non-limiting examples of genes that provide CHC-CoA include ansJ, ansK, ansL, chcA and ansM from the ansatrienin gene cluster of Streptomyces collinus (Chen et al., Eur. J. Biochem. 261:98, 1999) or plmJ, plmK, plmL, chcA and plmM from the phoslactomycin B gene cluster of Streptomyces sp. HK803 (Palaniappan et al., J. Biol. Chem. 278:35552, 2003) together with the chcB gene (Patton et al., Biochem. 39:7595, 2000) from S. collinus, S. avermitilis or S. coelicolor. The FabH, ACP and fabF genes can be expressed to allow initiation and elongation of co-cyclic fatty acids. Alternatively, the homologous genes can be isolated from microorganisms that make cyFA and expressed in C₁ metabolizing microorganisms of this disclosure.

The genes fabH, acp and fabF are sufficient to allow initiation and elongation of ω-cyclic fatty acids because they can have broad substrate specificity. If the coexpression of any of these genes with the ansJKLM/chcAB or pmlJKLM/chcAB genes does not yield cyFA, then fabH, acp or fabF homologs from microorganisms that make cyFAs can be isolated (e.g., by using degenerate PCR primers or heterologous DNA sequence probes) and co-expressed.

Fatty acids are a key intermediate in the production of fatty acid derivatives. The degree of saturation in fatty acid derivatives can be controlled by regulating the degree of saturation of the fatty acid intermediates. The sfa, gns, and fab families of genes can be expressed or over-expressed to control the saturation of fatty acids. Non-limiting examples of genes in these gene families that may be used in the present methods, and with C₁ metabolizing microorganisms of this disclosure, are listed in FIG. 1 of U.S. Pat. No. 8,283,143, which figure is herein incorporated by reference in its entirety.

C₁ metabolizing microorganisms of this disclosure can be engineered to produce unsaturated fatty acid derivatives by engineering the C₁ metabolizing microorganisms (e.g., methanotrophs) to over-express fabB, or by growing the C₁ metabolizing microorganism at low temperatures (e.g., less than 37° C.). In E. coli, FabB has preference to cisΔ³decenoyl-ACP and results in unsaturated fatty acid production. Over-expression of FabB results in the production of a significant percentage of unsaturated fatty acids (de Mendoza et al., J. Biol. Chem. 258:2098, 1983). A nucleic acid molecule encoding a fabB may be inserted into and expressed in C₁ metabolizing microorganisms (e.g., methanotrophs) not naturally having the gene. These unsaturated fatty acids can then be used as intermediates in C₁ metabolizing microorganisms that are engineered to produce fatty acid derivatives, such as fatty alcohols, fatty esters, waxes, hydroxy fatty acids, dicarboxylic acids, or the like.

Alternatively, a repressor of fatty acid biosynthesis, for example, fabR can be inhibited or deleted in C₁ metabolizing microorganisms (e.g., methanotrophs), which may also result in increased unsaturated fatty acid production as is seen in E. coli (Zhang et al., J. Biol. Chem. 277:15558, 2002). Further increase in unsaturated fatty acids may be achieved, for example, by over-expression of fabM (trans-2, cis-3-decenoyl-ACP isomerase) and controlled expression of fabK (trans-2-enoyl-ACP reductase II) from Streptococcus pneumoniae (Marrakchi et al., J. Biol. Chem. 277:44809, 2002), while deleting fabI (trans-2-enoyl-ACP reductase). Additionally, to increase the percentage of unsaturated fatty esters, a C₁ metabolizing microorganism (e.g., methanotroph) can also over-express fabB (encoding β-ketoacyl-ACP synthase I, Accession No. EC:2.3.1.41), sfa (encoding a suppressor of fabA), and gnsA and gnsB (both encoding secG null mutant suppressors, i.e., cold shock proteins). In some examples, an endogenous fabF gene can be attenuated, which can increase the percentage of palmitoleate (C_{16:1}) produced.

In another example, a desired fatty acid derivative is a hydroxylated fatty acid. Hydroxyl modification can occur throughout the chain using specific enzymes. In particular, ω-hydroxylation produces a particularly useful molecule containing functional groups at both ends of the molecule (e.g., allowing for linear polymerization to produce polyester plastics). In certain embodiments, a C₁ metabolizing microorganism (e.g., methanotroph) may comprise one or more modified CYP52A type cytochrome P450 selected from CYP52A13, CYP52A14, CYP52A17, CYP52A18, CYP52A12, and CYP52A12B, wherein the cytochrome modifies fatty acids into, for example, ω-hydroxy fatty acids. Different fatty acids are hydroxylated at different rates by different cytochrome P450s. To achieve efficient hydroxylation of a desired fatty acid feedstock, C₁ metabolizing microorganisms are generated to express one or more P450 enzymes that can ω-hydroxylate a wide range of highly abundant fatty acid substrates. Of particular interest are P450 enzymes that catalyze ω-hydroxylation of lauric acid (C_{12:0}), myristic acid (C₁₄:0), palmitic acid (C_{16:0}), stearic acid (C_{18:0}), oleic acid (C_{18:1}), linoleic acid (C_{18:2}), and α-linolenic acid (ω3, C_{18:3}). Examples of P450 enzymes with known ω-hydroxylation activity on different fatty acids that may be cloned into a C₁ metabolizing non-photosynthetic microorganism include CYP94A1 from Vicia sativa (Tijet et al., Biochem. J. 332:583, 1988); CYP 94A5 from Nicotiana tabacum (Le Bouquin et al., Eur. J. Biochem. 268:3083, 2001); CYP78A1 from Zea mays (Larkin, Plant Mol. Biol. 25:343, 1994); CYP 86A1 (Benveniste et al., Biochem. Biophys. Res. Commun. 243:688, 1998) and CYP86A8 (Wellesen et al., Proc. Nat'l. Acad. Sci. USA 98:9694, 2001) from Arabidopsis thaliana; CYP 92B1 from Petunia hybrida (Petkova-Andonova et al., Biosci. Biotechnol. Biochem. 66:1819, 2002); CYP102A1 (BM-3) mutant F87 from Bacillus megaterium (Oliver et al., Biochem. 36:1567, 1997); and CYP 4 family from mammal and insect (Hardwick, Biochem. Pharmacol. 75:2263, 2008).

In certain embodiments, a C₁ metabolizing non-photosynthetic microorganisms comprises a nucleic acid molecule encoding a P450 enzyme capable of introducing additional internal hydroxylation at specific sites of fatty acids or ω-hydroxy fatty acids, wherein the recombinant C₁ metabolizing microorganisms can produce internally oxidized fatty acids or ω-hydroxy fatty acids or aldehydes or dicarboxylic acids. Examples of P450 enzymes with known in-chain hydroxylation activity on different fatty acids that may be used in C₁ metabolizing microorganisms of this disclosure include CYP81B1 from Helianthus tuberosus with ω-1 to ω-5 hydroxylation (Cabello-Hurtado et al., J. Biol. Chem. 273:7260, 1998); CYP790C1 from Helianthus tuberosus with ω-1 and ω-2 hydroxylation (Kandel et al., J. Biol. Chem. 280:35881, 2005); CYP726A1 from Euphorbia lagscae with epoxidation on fatty acid unsaturation (Cahoon et al., Plant Physiol. 128:615, 2002); CYP152B1 from Sphingomonas paucimobilis with α-hydroxylation (Matsunaga et al., Biomed. Life Sci. 35:365, 2000); CYP2E1 and 4A1 from human liver with ω-1 hydroxylation (Adas et al., J. Lip. Res. 40:1990, 1999); P450_{BSβ} from Bacillus substilis with α- and β-hydroxylation (Lee et al., J. Biol. Chem. 278:9761, 2003); and CYP102A1 (BM-3) from Bacillus megaterium with ω-1, ω-2 and ω-3 hydroxylation (Shirane et al., Biochem. 32:13732, 1993).

In certain embodiments, a C₁ metabolizing non-photosynthetic microorganisms comprises a nucleic acid molecule encoding a P450 enzyme capable of modifying fatty acids to comprise a ω-hydroxylation can be further modified to further oxidize the ω-hydroxy fatty acid derivative to yield dicarboxylic acids. In many cases, a P450 enzyme capable of performing the hydroxylation in the first instance is also capable of performing further oxidation to yield a dicarboxylic acid. In other embodiments, non-specific native alcohol dehydrogenases in the host organism may oxidize the ω-hydroxy fatty acid to a dicarboxylic acid. In further embodiments, a C₁ metabolizing non-photosynthetic organism further comprises a nucleic acid molecule that encodes one or more fatty alcohol oxidases, (such as FAO1, FAO1B, FAO2, FAO2B) or alcohol dehydrogenases (such as ADH-A4, ADH-A4B, ADH-B4, ADH-B4B, ADH-A10 and ADH-B11) (e.g., from Candida tropicalis as listed in U.S. Patent Application Publication 2010/0291653, which list is incorporated herein in its entirety) to facilitate production of dicarboxylic acids.

The methods described herein permit production of fatty esters and fatty acid derivatives having varied carbon chain lengths. Chain length is controlled by thioesterase, which is produced by expression of the tes and fat gene families, and fatty acid elongase (i.e., KCS, KCR, HCD, and ECR). By expressing specific thioesterases, fatty acid derivatives having a desired carbon chain length for use as substrates of the fatty acid elongase can be produced. Non-limiting examples of suitable thioesterases are described herein and listed in U.S. Pat. No. 8,283,143 (FIG. 1, which figure is herein incorporated by reference). A nucleic acid molecule encoding a particular thioesterase may be introduced into a C₁ metabolizing microorganism (e.g., methanotroph) so that a fatty acid derivative of a particular carbon chain length is produced. In certain embodiments, expression of endogenous thioesterases are inhibited, suppressed, or down-regulated.

In certain embodiments, a fatty acid derivative has a carbon chain of about 8 to 24 carbon atoms, about 8 to 18 carbon atoms, about 10 to 18 carbon atoms, about 10 to 16 carbon atoms, about 12 to 16 carbon atoms, about 12 to 14 carbon atoms, about 14 to 24 carbon atoms, about 14 to 18 carbon atoms, about 8 to 16 carbon atoms, or about 8 to 14 carbon atoms. In alternative embodiments, a fatty acid derivative has a carbon chain of less than about 20 carbon atoms, less than about 18 carbon atoms, less than about 16 carbon atoms, less than about 14 carbon atoms, or less than about 12 carbon atoms. In other embodiments, a fatty ester product is a saturated or unsaturated fatty ester product having a carbon atom content between 8 and 24 carbon atoms. In further embodiments, a fatty ester product has a carbon atom content between 8 and 14 carbon atoms. In still further embodiments, a fatty ester product has a carbon content of 14 and 20 carbons. In yet other embodiments, a fatty ester is the methyl ester of C_{18:1}. In further embodiments, a fatty ester is the ethyl ester of C_{16:1}. In other embodiments, a fatty ester is the methyl ester of C_{16:1}. In yet other embodiments, a fatty ester is octadecyl ester of octanol.

Some microorganisms preferentially produce even- or odd-numbered carbon chain fatty acids and fatty acid derivatives. For example, E. coli normally produce even-numbered carbon chain fatty acids and fatty acid ethyl esters (FAEE). In certain embodiments, the methods disclosed herein may be used to alter that production in C₁ metabolizing microorganisms (e.g., methanotrophs) such that C₁ metabolizing microorganisms (e.g., methanotrophs) can be made to produce odd-numbered carbon chain fatty acid derivatives.

An ester includes what may be designated an “A” side and a “B” side. The B side may be contributed by a fatty acid produced from de novo synthesis in a C₁ metabolizing microorganism (e.g., methanotroph) of this disclosure. In some embodiments where a C₁ metabolizing microorganism (e.g., methanotroph) is additionally engineered to make alcohols, including fatty alcohols, the A side is also produced by a C₁ metabolizing microorganism (e.g., methanotroph). In yet other embodiments, the A side can be provided in the medium. By selecting a desired thioesterase encoding nucleic acid molecule, a B side (and an A side when fatty alcohols are being made) can be designed to be have certain carbon chain characteristics. These characteristics include points of branching, unsaturation, and desired carbon chain lengths.

When particular thioesterase and FAE genes are selected, the A and B side will have similar carbon chain characteristics when they are both contributed by a C₁ metabolizing microorganism (e.g., methanotroph) using fatty acid biosynthetic pathway intermediates. For example, at least about 50%, 60%, 70%, or 80% of the fatty esters produced will have A sides and B sides that vary by about 2, 4, 6, 8, 10, 12, or 14 carbons in length. The A side and the B side can also display similar branching and saturation levels.

In addition to producing fatty alcohols for contribution to the A side, a C₁ metabolizing microorganism (e.g., methanotroph) can produce other short chain alcohols, such as ethanol, propanol, isopropanol, isobutanol, and butanol for incorporation on the A side. For example, butanol can be made by a C₁ metabolizing microorganism (e.g., methanotroph). To create butanol producing cells, a C₁ metabolizing microorganism (e.g., methanotroph), for example, can be further engineered to express atoB (acetyl-CoA acetyltransferase) from Escherichia coli K12, β-hydroxybutyryl-CoA dehydrogenase from Butyrivibrio fibrisolvens, crotonase from Clostridium beijerinckii, butyryl CoA dehydrogenase from Clostridium beijerinckii, CoA-acylating aldehyde dehydrogenase (ALDH) from Cladosporium fulvum, and adhE encoding an aldehyde-alcohol dehydrogenase of Clostridium acetobutylicum in, for example, a pBAD24 expression vector under a prpBCDE promoter system. C₁ metabolizing microorganisms (e.g., methanotrophs) may be similarly modified to produce other short chain alcohols. For example, ethanol can be produced in a production host using the methods taught by Kalscheuer et al. (Microbiol. 152:2529, 2006).

### C₁ Metabolizing Microorganisms—Host Cells

The C₁ metabolizing microorganisms of the instant disclosure may be a natural strain, strain adapted (e.g., performing fermentation to select for strains with improved growth rates and increased total biomass yield compared to the parent strain), or recombinantly modified to produce very long carbon chain compounds of interest or to have increased growth rates or both (e.g., genetically altered to express a KCS, KCR, HCD, ECR, or a combination thereof). In certain embodiments, the C₁ metabolizing microorganisms are not photosynthetic microorganisms, such as algae or plants.

In certain embodiments, the present disclosure provides C₁ metabolizing microorganisms that are prokaryotes or bacteria, such as Methylomonas, Methylobacter, Methylococcus, Methylosinus, Methylocystis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, or Pseudomonas.

In further embodiments, the C₁ metabolizing bacteria are a methanotroph or a methylotroph. Exemplary methanotrophs include Methylomonas, Methylobacter, Methylococcus, Methylosinus, Methylocystis, Methylomicrobium, Methanomonas, Methylocella, or any combination thereof. Exemplary methylotrophs include Methylobacterium extorquens, Methylobacterium radiotolerans, Methylobacterium populi, Methylobacterium chloromethanicum, Methylobacterium nodulans, or any combination thereof.

In certain embodiments, methanotrophic bacteria are genetically engineered with the capability to convert C₁ substrate feedstock into very long carbon chain compounds. Methanotrophic bacteria have the ability to oxidize methane as a carbon and energy source. Methanotrophic bacteria are classified into three groups based on their carbon assimilation pathways and internal membrane structure: type I (gamma proteobacteria), type II (alpha proteobacteria, and type X (gamma proteobacteria). Type I methanotrophs use the ribulose monophosphate (RuMP) pathway for carbon assimilation whereas type II methanotrophs use the serine pathway. Type X methanotrophs use the RuMP pathway but also express low levels of enzymes of the serine pathway. Methanotrophic bacteria include obligate methanotrophs, which can only utilize C1 substrates for carbon and energy sources, and facultative methanotrophs, which naturally have the ability to utilize some multi-carbon substrates as a sole carbon and energy source.

Exemplary facultative methanotrophs include some species of Methylocella, Methylocystis, and Methylocapsa (e.g., Methylocella silvestris, Methylocella palustris, Methylocella tundrae, Methylocystis daltona strain SB2, Methylocystis bryophila, and Methylocapsa aurea KYG), Methylobacterium organophilum (ATCC 27,886), Methylibium petroleiphilum, or high growth variants thereof. Exemplary obligate methanotrophic bacteria include: Methylococcus capsulatus Bath, Methylomonas 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylomonas flagellata sp AJ-3670 (FERM P-2400), Methylacidiphilum infernorum and Methylomicrobium alcaliphilum, or a high growth variants thereof.

In still further embodiments, the present disclosure provides C₁ metabolizing microorganisms that are syngas metabolizing bacteria, such as Clostridium, Moorella, Pyrococcus, Eubacterium, Desulfobacterium, Carboxydothermus, Acetogenium, Acetobacterium, Acetoanaerobium, Butyribaceterium, Peptostreptococcus, or any combination thereof. Exemplary methylotrophs include Clostridium autoethanogenum, Clostridium ljungdahli, Clostridium ragsdalei, Clostridium carboxydivorans, Butyribacterium methylotrophicum, Clostridium woodii, Clostridium neopropanologen, or any combination thereof.

In any of the embodiments described herein, a C₁ metabolizing non-photosynthetic microorganism is not a yeast, such as Yarrowia.

In certain other embodiments, the C₁ metabolizing non-photosynthetic microorganism is an obligate C₁ metabolizing non-photosynthetic microorganism, such as an obligate methanotroph or methylotroph. In further embodiments, the C₁ metabolizing non-photosynthetic microorganism is a recombinant microorganism comprising a heterologous polynucleotide encoding a KCS, a KCR, an HCD, an ECR, a combination thereof, or all four.

### C₁ Metabolizing Microorganisms—Non-Natural or Recombinant

In some embodiments, as described herein, there are provided recombinant C₁ metabolizing microorganisms (e.g., non-natural methanotroph bacteria) having a β-ketoacyl-CoA synthase, a β-hydroxy acyl-CoA dehydratase, a β-ketoacyl-CoA reductase, and an enoyl-CoA reductase that utilize a C₁ substrate feedstock (e.g., methane) to generate >C₂₄ very long carbon chain compounds, such as very long chain fatty acyl-CoA. In various embodiments, a recombinant C₁ metabolizing microorganism expresses or over expresses a nucleic acid molecule that encodes a KCS enzyme. In certain embodiments, a KCS enzyme may be endogenous to the C₁ metabolizing microorganism or a KCS enzyme may be heterologous to the C₁ metabolizing microorganism.

In one aspect, the present disclosure provides a non-natural methanotroph having a recombinant nucleic acid molecules encoding the following enzymes: (i) a β-ketoacyl-CoA synthase, (ii) a β-hydroxy acyl-CoA dehydratase, (iii) a β-ketoacyl-CoA reductase, and (iv) an enoyl-CoA reductase, wherein the methanotroph is capable of converting a C₁ substrate into a very long carbon chain compound selected from a very long chain fatty acyl-CoA, a very long chain fatty aldehyde, a very long chain fatty alcohol (primary or secondary), a very long chain fatty ester wax, a very long chain alkane, a very long chain ketone, or a combination thereof. In certain embodiments, the KCS is CER6, Elo1, Fen1/Elo2, Sur4/Elo3, KCS1, KCS2, KCS11, KCS20, KCS9, ELOVL1, ELOVL2, ELOVL3, ELOVL4, ELOVL5, ELOVL6, ELOVL7, or FDH. In certain embodiments, the non-natural methanotroph comprises recombinant nucleic acid molecules encoding at least two different KCS enzymes. In certain embodiments, the KCR is CER10, KAR, GL8A, GL8B, Ybr159w, AYR1, or At1g67730. In certain embodiments, the HCD is PHS1, PAS2, HACD1, HACD2, HACD3, HACD4, or PAS2-1. In certain embodiments, the ECR is CER10, TER, TSC13, or GhECR1, GhECR2.

In certain embodiments, the non-natural methanotroph further comprises a recombinant nucleic acid encoding a fatty alcohol forming acyl-CoA reductase capable of forming a very long chain fatty alcohol. In certain embodiments, as the fatty alcohol forming acyl-CoA reductase is FAR, CER4 (Genbank Accession No. JN315781.1), or Maqu_2220. In certain embodiments, the non-natural methanotroph further comprises recombinant nucleic acid molecules encoding a fatty acyl-CoA reductase capable of forming a very long chain fatty aldehyde and an aldehyde reductase capable of forming a very long chain fatty alcohol. In certain embodiments, the fatty acyl-CoA reductase is ACR1 or CER3. In certain embodiments, the aldehyde reductase is YqhD. In some embodiments, the process will result in the production of fatty alcohols comprising greater than C₂₄ carbons in length.

In certain embodiments, the non-natural methanotroph further comprises recombinant nucleic acid molecules encoding a fatty alcohol forming acyl-CoA reductase capable of forming a very long chain fatty alcohol and an ester synthase capable of forming a very long chain fatty ester wax. In certain embodiments, the fatty alcohol forming acyl-CoA reductase is FAR, CER4 (Genbank Accession No. JN315781.1), or Maqu_2220. In certain embodiments, the ester synthase is WSD1. In some embodiments, the process will result in the production of fatty ester waxes comprising greater than C24 carbons in length.

In certain embodiments, the non-natural methanotroph further comprises recombinant nucleic acid molecules encoding a fatty acyl-CoA reductase capable of forming a very long chain fatty aldehyde and an aldehyde decarbonylase capable of forming a very long chain alkane. In certain embodiments, the fatty acyl-CoA reductase is ACR1 or CER3. In certain embodiments, the aldehyde decarbonylase is CER1 or CER22. In some embodiments, the process will result in the production of very long chain alkanes comprising greater than C24 carbons in length.

In certain embodiments, the non-natural methanotroph further comprises recombinant nucleic acid molecules encoding a fatty acyl-CoA reductase capable of forming a very long chain fatty aldehyde, an aldehyde decarbonylase capable of forming a very long chain alkane, and an alkane hydroxylase capable of forming a very long chain fatty secondary alcohol, and an alcohol dehydrogenase capable of forming a very long chain ketone. In certain embodiments, the fatty acyl-CoA reductase is ACR1 or CER3. In certain embodiments, the aldehyde decarbonylase is CER1 or CER22. In certain embodiments, the alkane hydroxylase and alcohol dehydrogenase is MAH1.

In any of the aforementioned recombinant C₁ metabolizing microorganisms capable of producing very long carbon chain compounds as encompassed by the present disclosure, the non-natural methanotrophs further comprise a recombinant nucleic acid molecule encoding a thioesterase, such as a tesA lacking a leader sequence, UcFatB, or BTE. In certain embodiments, the endogenous thioesterase activity is reduced, minimal or abolished as compared to unaltered endogenous thioesterase activity.

In any of the aforementioned recombinant C₁ metabolizing microorganisms capable of producing very long carbon chain compounds as encompassed by the present disclosure, the non-natural methanotrophs further comprise a recombinant nucleic acid molecule encoding an acyl-CoA synthetase, such as FadD, yng1, or FAA2. In certain embodiments, the endogenous acyl-CoA synthetase activity is reduced, minimal or abolished as compared to unaltered endogenous acyl-CoA synthetase activity.

In further embodiments, the present disclosure provides a non-natural methanotroph having recombinant nucleic acid molecules encoding heterologous KCS, KCR, HCD, and ECR, a recombinant nucleic acid molecule encoding a heterologous thioesterase, and a recombinant nucleic acid molecule encoding a heterologous acyl-CoA synthetase, wherein the methanotroph is capable of converting a C₁ substrate into a very long chain acyl-CoA. In certain embodiments, wherein the non-natural methanotroph further comprises a nucleic acid molecule encoding a fatty acyl-CoA reductase or fatty alcohol forming acyl-CoA reductase, the fatty acyl-CoA reductase or fatty alcohol forming acyl-CoA reductase is over-expressed in the non-natural methanotroph as compared to the expression level of the native fatty acyl-CoA reductase or fatty alcohol forming acyl-CoA reductase, respectively. In certain embodiments, the fatty acyl-CoA reductase capable of forming a fatty aldehyde is ACR1 or CER3, or the fatty alcohol forming acyl-CoA reductase capable of forming a fatty alcohol is FAR, CER4, or Maqu_2220. In certain embodiments, the acyl-CoA synthetase is FadD, yng1, or FAA2.

Any of the aforementioned recombinant C₁ metabolizing microorganisms (e.g., non-natural methanotroph bacteria) may have a FAR enzyme or functional fragment thereof can be derived or obtained from a species of Marinobacter, such as M. algicola, M. alkaliphilus, M. aquaeolei, M. arcticus, M. bryozoorum, M. daepoensis, M. excellens, M. flavimaris, M. guadonensis, M. hydrocarbonoclasticus, M. koreenis, M. lipolyticus, M. litoralis, M. lutaoensis, M. maritimus, M. sediminum, M. squalenivirans, M. vinifirmus, or equivalent and synonymous species thereof. In certain embodiments, a FAR enzyme for use in the compositions and methods disclosed herein is from marine bacterium Marinobacter algicola DG893 (Genbank Accession No. EDM49836.1, FAR “Maa_893”) or Marinobacter aquaeolei VT8 (Genbank Accession No. YP_959486.1, FAR “Maqu_2220”) or Oceanobacter sp. RED65 (Genbank Accession No. EAT13695.1, FAR “Ocs_65”).

In still further embodiments of any of the aforementioned recombinant C₁ metabolizing microorganisms (e.g., non-natural methanotroph bacteria), a FAR enzyme or functional fragment thereof is FAR_Hch (Hahella chejuensis KCTC 2396, GenBank Accession No. YP_436183.1); FAR_Act (from marine Actinobacterium strain PHSC20C1, GenBank Accession No. EAR25464.1), FAR_Mme (marine metagenome, GenBank Accession No. EDD40059.1), FAR_Aec (Acromyrmex echinatior, GenBank Accession No. EGI61731.1), FAR_Cfl (Camponotus floridanus, GenBank Accession No. EFN62239.1), and FAR_Sca (Streptomyces cattleya NRRL 8057, GenBank Accession No. YP_006052652.1). In other embodiments, a FAR enzyme or functional fragment thereof is isolated or derived from Vitis vinifera (FAR_Vvi, GenBank Accession No. CAO22305.1 or CAO67776.1), Desulfatibacillum alkenivorans AK-01 (FAR_Dal, GenBank Accession No. YP_002430327.1), Simmondsia chinensis (FAR_Sch, GenBank Accession No. AAD38039.1), Bombyx mori (FAR_Bmo, GenBank Accession No. BAC79425.1), Arabidopsis thaliana (FAR_Ath; GenBank Accession No. DQ446732.1 or NM_115529.1), or Ostrinia scapulalis (FAR_Osc; GenBank Accession no. EU817405.1).

In certain embodiments, a FAR enzyme or functional fragment thereof is derived or obtained from M. algicola DG893 or Marinobacter aquaeolei YT8 and has an amino acid sequence that is at least at least 75%, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to the sequence set forth in Genbank Accession No. EDM49836.1 or YP_959486.1, respectively, or a functional fragment thereof. In another embodiment, the recombinant encoded FAR enzyme has an amino acid sequence that is identical to the sequence set forth in Genbank Accession No. EDM49836.1 or YP_959486.1.

In another aspect, this disclosure provides any of the aforementioned C₁ metabolizing microorganism or non-natural methanotrophs further comprise a recombinant nucleic acid molecule encoding a P450 enzyme or monoxygenase enzyme to produce an ω-hydroxy fatty acid. In certain embodiments, the endogenous alcohol dehydrogenase activity is inhibited as compared to unaltered endogenous alcohol dehydrogenase activity. In other embodiments, the endogenous alcohol dehydrogenase activity is increased or elevated as compared to unaltered endogenous alcohol dehydrogenase activity to produce dicarboxylic acid.

In any of the aforementioned non-natural methanotrophs, a very long carbon chain compound is produced comprising one or more of about C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, C₁₈₁-C₂₀₀, C₂₅-C₄₀, C₂₅-C₅₀, C₂₅-C₇₅, C₂₅-C₁₀₀, C₂₅-C₁₂₅, C₂₅-C₁₅₀, C₂₅-C₁₇₅, or C₂₅-C₂₀₀ very long carbon chain compounds. In certain embodiments, the methanotroph produces very long fatty alcohol comprising C₂₅ to C₅₀ very long fatty chain alcohol and the C₂₅ to C₅₀ very long fatty chain alcohols comprise at least 70% of the total fatty alcohol. In further embodiments, the methanotroph produces a very long fatty alcohol comprising a very long branched chain fatty alcohol. In certain embodiments, the methanotroph produces very long chain wax ester comprising C₂₅ to C₅₀ very long fatty wax ester and the C₂₅ to C₅₀ very long chain wax esters comprise at least 70% of the total wax ester. In certain embodiments, the methanotroph produces very long chain alkane comprising C₂₅ to C₅₀ very long chain alkane and the C₂₅ to C₅₀ very long chain alkanes comprise at least 70% of the total alkanes. In certain embodiments, the methanotroph produces very long chain ketone comprising C₂₅ to C₅₀ very long chain ketone and the C₂₅ to C₅₀ very long chain ketones comprise at least 70% of the total ketone.

In any of the aforementioned non-natural methanotrophs, the amount of very long chain fatty acyl-CoA, very long chain fatty aldehyde, very long chain fatty alcohol, very long chain fatty ester wax, very long chain alkane or very long chain ketone produced by the non-natural methanotroph ranges from about 1 mg/L to about 500 g/L. In certain other embodiments, a C₁ substrate feedstock for a C₁ metabolizing microorganism or non-natural methanotroph as described is methane, methanol, formaldehyde, formic acid or a salt thereof, carbon monoxide, carbon dioxide, a methylamine, a methylthiol, a methylhalogen, natural gas, or unconventional natural gas. In certain embodiments, a C₁ metabolizing microorganism or non-natural methanotroph is capable of converting natural gas, unconventional natural gas or syngas (or syngas comprising methane) into a greater than C₂₄ very long fatty acyl-CoA, very long fatty aldehyde, very long fatty alcohol, very long chain fatty ester wax, very long chain alkane, or very long chain ketone.

In any of the aforementioned C₁ metabolizing microorganisms or non-natural methanotrophs, the host methanotroph can be Methylococcus capsulatus Bath strain, Methylomonas 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylobacterium organophilum (ATCC 27,886), Methylomonas sp AJ-3670 (FERM P-2400), Methylocella silvestris, Methylocella palustris (ATCC 700799), Methylocella tundrae, Methylocystis daltona strain SB2, Methylocystis bryophila, Methylocapsa aurea KYG, Methylacidiphilum infernorum, Methylibium petroleiphilum, Methylomicrobium alcaliphilum, or a combination thereof.

Any of the aforementioned C₁ metabolizing microorganisms or non-natural methanotroph bacteria may also have undergone strain adaptation under selective conditions to produce variants with improved properties for fatty acid derivative production, before or after introduction of the recombinant nucleic acid molecules. Improved properties may include increased growth rate, yield of desired products (e.g., very long chain carbon compounds), or tolerance to process or culture contaminants. In particular embodiments, a high growth variant C₁ metabolizing microorganism or methanotroph comprises a host bacteria that is capable of growing on a methane feedstock as a primary carbon and energy source and that possesses a faster exponential phase growth rate (i.e., shorter doubling time) than its parent, reference, or wild-type bacteria (see, e.g., U.S. Pat. No. 6,689,601).

Each of the microorganisms of this disclosure may be grown as an isolated culture, with a heterologous organism that may aid with growth, or one or more of these bacteria may be combined to generate a mixed culture. In still further embodiments, C₁ metabolizing non-photosynthetic microorganisms of this disclosure are obligate C₁ metabolizing non-photosynthetic microorganisms.

### C₁ Metabolizing Microorganisms—Producing Very Long Carbon Chain Compounds

In another aspect, as described herein, there are provided methods for making very long carbon chain compounds by culturing a non-natural C₁ metabolizing non-photosynthetic microorganism with a C₁ substrate feedstock and recovering the very long carbon chain compounds, wherein the C₁ metabolizing non-photosynthetic microorganism comprises one or more recombinant nucleic acid molecules encoding a β-ketoacyl-CoA synthase, a β-ketoacyl-CoA reductase, a β-hydroxy acyl-CoA dehydratase, and an enoyl-CoA reductase, and wherein the C₁ metabolizing non-photosynthetic microorganism converts the C₁ substrate into a greater than C₂₄ very long carbon chain compound comprising a very long chain acyl-CoA, a very long chain fatty aldehyde, a very long chain fatty alcohol (primary or secondary), a very long chain alkane, a very long chain ketone, or a combination thereof.

In certain embodiments, the C₁ metabolizing non-photosynthetic microorganism being cultured is Methylomonas, Methylobacter, Methylococcus, Methylosinus, Methylocystis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, or Pseudomonas. In further embodiments, C₁ metabolizing non-photosynthetic microorganism being cultured is bacteria, such as a methanotroph or methylotroph.

The methanotroph may be a Methylomonas sp. 16a (ATCC PTA 2402), Methylosinus trichosporium (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus (NRRL B-11,201), Methylobacterium organophilum (ATCC 27,886), Methylomonas sp. AJ-3670 (FERM P-2400), Methylocella silvestris, Methylacidiphilum infernorum, Methylibium petroleiphilum, or a combination thereof. In certain embodiments, the methanotroph culture further comprises one or more heterologous bacteria.

The methylotroph may be a Methylobacterium extorquens, Methylobacterium radiotolerans, Methylobacterium populi, Methylobacterium chloromethanicum, Methylobacterium nodulans, or a combination thereof.

In further embodiments, the C₁ metabolizing microorganism or bacteria can metabolize natural gas, unconventional natural gas, or syngas. In certain embodiments, the syngas metabolizing bacteria include Clostridium autoethanogenum, Clostridium ljungdahli, Clostridium ragsdalei, Clostridium carboxydivorans, Butyribacterium methylotrophicum, Clostridium woodii, Clostridium neopropanologen, or a combination thereof. In certain other embodiments, the metabolizing non-photosynthetic microorganism is an obligate C₁ metabolizing non-photosynthetic microorganism. In certain other embodiments, the metabolizing non-photosynthetic microorganism is an facultative C₁ metabolizing non-photosynthetic microorganism.

In any of the aforementioned methods, the cultured C₁ metabolizing microorganism contains a fatty alcohol forming fatty acyl-CoA reductase, such as FAR, CER4 (Genbank Accession No. JN315781.1), or Maqu_2220, capable of forming a very long chain fatty alcohol. In certain embodiments, the C₁ metabolizing microorganism being cultured contains a fatty acyl-CoA reductase capable of forming a fatty aldehyde, such as acr1 or CER3. In some embodiments, the process will result in the production of fatty alcohols comprising greater than C₂₄ carbons in length.

In any of the aforementioned recombinant C₁ metabolizing microorganisms capable of producing very long carbon chain compounds as encompassed by the present methods, the C₁ metabolizing microorganisms further comprise a recombinant nucleic acid molecule encoding a thioesterase, such as a tesA lacking a leader sequence, UcFatB, or BTE. In certain embodiments, the endogenous thioesterase activity is reduced, minimal or abolished as compared to unaltered endogenous thioesterase activity.

In any of the aforementioned recombinant C₁ metabolizing microorganisms capable of producing very long carbon chain compounds as encompassed by the present methods, the C₁ metabolizing microorganisms further comprise a recombinant nucleic acid molecule encoding an acyl-CoA synthetase, such as FadD, yng 1, or FAA2. In certain embodiments, the endogenous acyl-CoA synthetase activity is reduced, minimal or abolished as compared to unaltered endogenous acyl-CoA synthetase activity.

In further embodiments, the present methods provide a C₁ metabolizing microorganism having a recombinant nucleic acid encoding heterologous KCS, a recombinant nucleic acid encoding heterologous KCR, a recombinant nucleic acid encoding heterologous HCD, a recombinant nucleic acid encoding heterologous ECR, a recombinant nucleic acid molecule encoding a heterologous fatty alcohol forming acyl-CoA reductase, a recombinant nucleic acid molecule encoding a heterologous thioesterase, and a recombinant nucleic acid molecule encoding a heterologous acyl-CoA synthetase, wherein the C₁ metabolizing microorganism is capable of converting a C₁ substrate into a greater than C₂₄ fatty alcohol. In certain embodiments, the fatty alcohol forming acyl-CoA reductase is over-expressed in the cultured C₁ metabolizing microorganism as compared to the expression level of the native fatty alcohol forming acyl-CoA reductase. In certain embodiments, the fatty alcohol forming acyl-CoA reductase capable of forming a fatty alcohol is FAR, CER4, or Maqu_2220. In certain embodiments, the acyl-CoA synthetase is FadD, yng 1, or FAA2.

In any of the aforementioned cultured C1 metabolizing microorganisms, the methods produce a very long carbon chain compound comprising one or more of C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, C₁₈₁-C₂₀₀, C₂₅-C₄₀, C₂₅-C₅₀, C₂₅-C₇₅, C₂₅-C₁₀₀, C₂₅-C₁₂₅, C₂₅-C₁₅₀, C₂₅-C₁₇₅, or C₂₅-C₂₀₀ very long carbon chain compounds. In certain embodiments, the C1 metabolizing microorganisms produce very long fatty alcohol comprising C₂₅ to C₅₀ very long fatty chain alcohol and the C₂₅ to C₅₀ very long fatty chain alcohols comprise at least 70% of the total fatty alcohol. In further embodiments, the methanotroph produces a very long fatty alcohol comprising a very long branched chain fatty alcohol. In certain embodiments, the C1 metabolizing microorganisms produce very long chain wax ester comprising C₂₅ to C₅₀ very long fatty wax ester and the C₂₅ to C₅₀ very long chain wax esters comprise at least 70% of the total wax ester. In certain embodiments, the C1 metabolizing microorganisms produce very long chain alkane comprising C₂₅ to C₅₀ very long chain alkane and the C₂₅ to C₅₀ very long chain alkanes comprise at least 70% of the total alkanes. In certain embodiments, the C1 metabolizing microorganisms produce very long chain ketone comprising C₂₅ to C₅₀ very long chain ketone and the C₂₅ to C₅₀ very long chain ketones comprise at least 70% of the total ketone.

In any of the aforementioned cultured C₁ metabolizing microorganism, the amount of very long chain fatty acyl-CoA, very long chain fatty aldehyde, very long chain fatty alcohol, very long chain wax ester, very long chain alkane, or very long chain ketone produced by the C₁ metabolizing microorganisms range from about 1 mg/L to about 500 g/L. In certain other embodiments, the C₁ substrate feedstock for the C₁ metabolizing microorganisms used in the methods of making very long carbon chain compounds is methane, methanol, formaldehyde, formic acid or a salt thereof, carbon monoxide, carbon dioxide, a methylamine, a methylthiol, a methylhalogen, natural gas, or unconventional natural gas. In certain embodiments, the C₁ metabolizing microorganisms convert natural gas, unconventional natural gas or syngas comprising methane into a greater than C₂₄ acyl-CoA, fatty aldehyde, fatty alcohol, wax ester, alkane, or ketone.

In any of the aforementioned methods, the C₁ metabolizing microorganisms can be cultured in a controlled culturing unit, such as a fermentor or bioreactor.

### Codon Optimization

Expression of recombinant proteins is often difficult outside their original host. For example, variation in codon usage bias has been observed across different species of bacteria (Sharp et al., Nucl. Acids. Res. 33:1141, 2005). Over-expression of recombinant proteins even within their native host may also be difficult. In certain embodiments of the invention, nucleic acids (e.g., nucleic acids encoding fatty acid elongation enzymes) that are to be introduced into host methanotrophic bacteria as described herein may undergo codon optimization to enhance protein expression. Codon optimization refers to alteration of codons in genes or coding regions of nucleic acids for transformation of a methanotrophic bacterium to reflect the typical codon usage of the host bacteria species without altering the polypeptide for which the DNA encodes. Codon optimization methods for optimum gene expression in heterologous hosts have been previously described (see, e.g., Welch et al., PLoS One 4:e7002, 2009; Gustafsson et al., Trends Biotechnol. 22:346, 2004; Wu et al., Nucl. Acids Res. 35:D76, 2007; Villalobos et al., BMC Bioinformatics 7:285, 2006; U.S. Patent Application Publication Nos. US 2011/0111413; US 2008/0292918; disclosure of which are incorporated herein by reference, in their entirety).

### Transformation Methods

Any of the recombinant C₁ metabolizing microorganisms or methanotrophic bacteria described herein may be transformed to comprise at least one exogenous nucleic acid to provide the host bacterium with a new or enhanced activity (e.g., enzymatic activity) or may be genetically modified to remove or substantially reduce an endogenous gene function using a variety of methods known in the art.

Transformation refers to the transfer of a nucleic acid (e.g., exogenous nucleic acid) into the genome of a host cell, resulting in genetically stable inheritance. Host cells containing the transformed nucleic acid molecules are referred to as “non-naturally occurring” or “recombinant” or “transformed” or “transgenic” cells.

Expression systems and expression vectors useful for the expression of heterologous nucleic acids in C₁ metabolizing microorganisms or methanotrophic bacteria are known.

Electroporation of C₁ metabolizing bacteria has been previously described in Toyama et al., FEMS Microbiol. Lett. 166:1, 1998; Kim and Wood, Appl. Microbiol. Biotechnol. 48:105, 1997; Yoshida et al., Biotechnol. Lett. 23:787, 2001, and U.S. Patent Application Publication No. US 2008/0026005.

Bacterial conjugation, which refers to a particular type of transformation involving direct contact of donor and recipient cells, is more frequently used for the transfer of nucleic acids into C₁ metabolizing bacteria. Bacterial conjugation involves mixing “donor” and “recipient” cells together in close contact with each other. Conjugation occurs by formation of cytoplasmic connections between donor and recipient bacteria, with unidirectional transfer of newly synthesized donor nucleic acid molecules into the recipient cells. A recipient in a conjugation reaction is any cell that can accept nucleic acids through horizontal transfer from a donor bacterium. A donor in a conjugation reaction is a bacterium that contains a conjugative plasmid, conjugative transposon, or mobilized plasmid. The physical transfer of the donor plasmid can occur through a self-transmissible plasmid or with the assistance of a “helper” plasmid. Conjugations involving C₁ metabolizing bacteria have been previously described in Stolyar et al., Mikrobiologiya 64:686, 1995; Motoyama et al., Appl. Micro. Biotech. 42:67, 1994; Lloyd et al., Arch. Microbiol. 171:364, 1999; and Odom et al., PCT Publication No. WO 02/18617; Ali et al., Microbiol. 152:2931, 2006.

Expression of heterologous nucleic acids in C1 metabolizing bacteria is known in the art (see, e.g., U.S. Pat. No. 6,818,424; U.S. Patent Application Publication No. US 2003/0003528). Mu transposon based transformation of methylotrophic bacteria has been described (Akhverdyan et al., Appl. Microbiol. Biotechnol. 91:857, 2011). A mini-Tn7 transposon system for single and multicopy expression of heterologous genes without insertional inactivation of host genes in Methylobacterium has been described (U.S. Patent Application Publication No. US 2008/0026005).

Various methods for inactivating, knocking-out, or deleting endogenous gene function in C₁ metabolizing bacteria may be used. Allelic exchange using suicide vectors to construct deletion/insertional mutants in slow growing C₁ metabolizing bacteria have also been described in Toyama and Lidstrom, Microbiol. 144:183, 1998; Stolyar et al., Microbiol. 145:1235, 1999; Ali et al., Microbiol. 152:2931, 2006; Van Dien et al., Microbiol. 149:601, 2003.

Suitable homologous or heterologous promoters for high expression of exogenous nucleic acids may be utilized. For example, U.S. Pat. No. 7,098,005 describes the use of promoters that are highly expressed in the presence of methane or methanol for heterologous gene expression in C₁ metabolizing bacteria. Additional promoters that may be used include deoxy-xylulose phosphate synthase methanol dehydrogenase operon promoter (Springer et al., FEMS Microbiol. Lett. 160:119, 1998); the promoter for PHA synthesis (Foellner et al., Appl. Microbiol. Biotechnol. 40:284, 1993); or promoters identified from a native plasmid in methylotrophs (European Patent No. EP 296484). Non-native promoters include the lac operon Plac promoter (Toyama et al., Microbiol. 143:595, 1997) or a hybrid promoter such as Ptrc (Brosius et al., Gene 27:161, 1984). In certain embodiments, promoters or codon optimization are used for high constitutive expression of exogenous nucleic acids encoding glycerol utilization pathway enzymes in host methanotrophic bacteria. Regulated expression of an exogenous nucleic acid in the host methanotrophic bacterium may also be utilized. In particular, regulated expression of exogenous nucleic acids encoding glycerol utilization enzymes may be desirable to optimize growth rate of the non-naturally occurring methanotrophic bacteria. It is possible that in the absence of glycerol (e.g., during growth on methane as a carbon source), for the glycerol utilization pathway to run in reverse, resulting in secretion of glycerol from the bacteria, thereby lowering growth rate. Controlled expression of nucleic acids encoding glycerol utilization pathway enzymes in response to the presence of glycerol may optimize bacterial growth in a variety of carbon source conditions. For example, an inducible/regulatable system of recombinant protein expression in methylotrophic and methanotrophic bacteria, as described in U.S. Patent Application Publication No. US 2010/0221813, may be used. Regulation of glycerol utilization genes in bacteria is well established (Schweizer and Po, J. Bacteriol. 178:5215, 1996; Abram et al., Appl. Environ. Microbiol. 74:594, 2008; Darbon et al., Mol. Microbiol. 43:1039, 2002; Weissenborn et al., J. Biol. Chem. 267:6122, 1992). Glycerol utilization regulatory elements may also be introduced or inactivated in host methanotrophic bacteria for desired expression levels of exogenous nucleic acid molecules encoding glycerol utilization pathway enzymes.

Methods of screening are disclosed in Brock, supra. Selection methods for identifying allelic exchange mutants are known in the art (see, e.g., U.S. Patent Appl. Publication No. US 2006/0057726, Stolyar et al., Microbiol. 145:1235, 1999; and Ali et al., Microbiol. 152:2931, 2006.

### Culture Methods

A variety of culture methodologies may be used for recombinant methanotrophic bacteria described herein. For example, methanotrophic bacteria may be grown by batch culture or continuous culture methodologies. In certain embodiments, the cultures are grown in a controlled culture unit, such as a fermentor, bioreactor, hollow fiber membrane bioreactor, or the like.

A classical batch culturing method is a closed system where the composition of the media is set at the beginning of the culture and not subject to external alterations during the culture process. Thus, at the beginning of the culturing process, the media is inoculated with the desired C₁ metabolizing microorganism (e.g., methanotroph) and growth or metabolic activity is permitted to occur without adding anything to the system. Typically, however, a “batch” culture is batch with respect to the addition of carbon source and attempts are often made at controlling factors such as pH and oxygen concentration. In batch systems, the metabolite and biomass compositions of the system change constantly up to the time the culture is terminated. Within batch cultures, cells moderate through a static lag phase to a high growth logarithmic phase and finally to a stationary phase where growth rate is diminished or halted. If untreated, cells in the stationary phase will eventually die. Cells in logarithmic growth phase are often responsible for the bulk production of end product or intermediate in some systems. Stationary or post-exponential phase production can be obtained in other systems.

The Fed-Batch system is a variation on the standard batch system. Fed-Batch culture processes comprise a typical batch system with the modification that the substrate is added in increments as the culture progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measureable factors, such as pH, dissolved oxygen, and the partial pressure of waste gases such as CO₂. Batch and Fed-Batch culturing methods are common and known in the art (see, e.g., Thomas D. Brock, Biotechnology: A Textbook of Industrial Microbiology, 2^{nd} Ed. (1989) Sinauer Associates, Inc., Sunderland, Mass.; Deshpande, Appl. Biochem. Biotechnol. 36:227, 1992).

Continuous cultures are “open” systems where a defined culture media is added continuously to a bioreactor and an equal amount of conditioned media is removed simultaneously for processing. Continuous cultures generally maintain the cells at a constant high liquid phase density where cells are primarily in logarithmic phase growth. Alternatively, continuous culture may be practiced with immobilized cells where carbon and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method will maintain a limited nutrient, such as the carbon source or nitrogen level, at a fixed rate and allow all other parameters to modulate. In other systems, a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art, and a variety of methods are detailed by Brock, supra.

### Very Long Carbon Chain Compound Compositions

By way of background, stable isotopic measurements and mass balance approaches are widely used to evaluate global sources and sinks of methane (see Whiticar and Faber, Org. Geochem. 10:759, 1986; Whiticar, Org. Geochem. 16: 531, 1990). To use δ¹³C values of residual methane to determine the amount oxidized, it is necessary to know the degree of isotopic fractionation caused by microbial oxidation of methane. For example, aerobic methanotrophs can metabolize methane through a specific enzyme, methane monoxygenase (MMO). Methanotrophs convert methane to methanol and subsequently formaldehyde. Formaldehyde can be further oxidized to CO₂ to provide energy to the cell in the form of reducing equivalents (NADH), or incorporated into biomass through either the RuMP or Serine cycles (Hanson and Hanson, Microbiol. Rev. 60:439, 1996), which are directly analogous to carbon assimilation pathways in photosynthetic organisms.

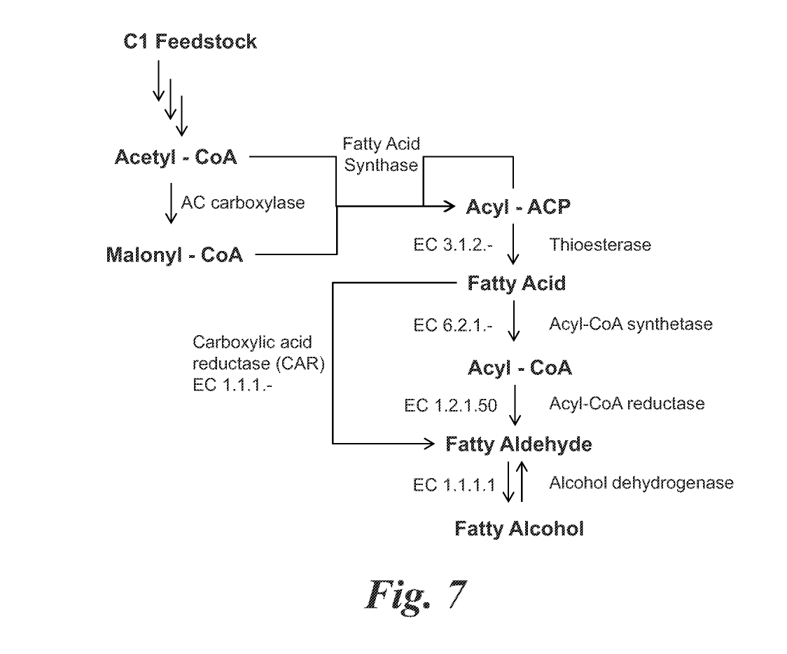

More specifically, a Type I methanotroph uses the RuMP pathway for biomass synthesis and generates biomass entirely from CH₄, whereas a Type II methanotroph uses the serine pathway that assimilates 50-70% of the cell carbon from CH₄ and 30-50% from CO₂ (Hanson and Hanson, 1996). Methods for measuring carbon isotope compositions are provided in, for example, Templeton et al. (Geochim. Cosmochim. Acta 70:1739, 2006), which methods are hereby incorporated by reference in their entirety. The ¹³C/¹²C stable carbon ratio of an oil composition from a biomass (provided as a “delta” value ‰, δ¹³C) can vary depending on the source and purity of the C₁ substrate used (see, e.g., FIG. 7).

Very long carbon chain compound compositions produced using a C₁ metabolizing non-photosynthetic microorganisms and methods described herein, may be distinguished from very long carbon chain compounds produced from petrochemicals or from photosynthetic microorganisms or plants by carbon fingerprinting. In certain embodiments, compositions of greater than C₂₄ fatty acyl-CoA, fatty aldehyde, fatty alcohol, fatty ester wax, alkane, ketone, or any combination thereof have a δ¹³C of less than −30‰, less than −31‰, less than −32‰, less than −33‰, less than −34‰, less than −35‰, less than −36‰, less than −37‰, less than −38‰, less than −39‰, less than −40‰, less than −41%, less than −42‰, less than −43‰, less than −44‰, less than −45‰, less than −46‰, less than −47‰, less than −48‰, less than −49‰, less than −50‰, less than −51‰, less than −52‰, less than −53‰, less than −54‰, less than −55‰, less than −56‰, less than −57‰, less than −58‰, less than −59‰, less than −60‰, less than −61‰, less than −62‰, less than −63‰, less than −64‰, less than −65‰, less than −66‰, less than −67‰, less than −68‰, less than −69‰, or less than −70‰.

In some embodiments, a C₁ metabolizing microorganism biomass comprises a very long carbon chain compound composition as described herein, wherein the very long carbon chain compound containing biomass or a very long carbon chain compound composition has a δ¹³C of about −35‰ to about −50‰, −45‰ to about −35‰, or about −50‰ to about −40‰, or about −45‰ to about −65‰, or about −60‰ to about −70‰, or about −30‰ to about −70‰. In certain embodiments, a very long carbon chain compound composition comprises at least 50% very long carbon chain compound. In further embodiments, a very long carbon chain compound composition comprises a very long chain fatty acyl-CoA, a very long chain fatty aldehyde, a very long chain fatty alcohol, a very long chain fatty ester wax, a very long chain alkane, a very long chain ketone, or any combination thereof. In still further embodiments, a very long chain carbon compound composition comprises C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, C₁₈₁-C₂₀₀, C₂₅-C₄₀, C₂₅-C₅₀, C₂₅-C₇₅, C₂₅-C₁₀₀, C₂₅-C₁₂₅, C₂₅-C₁₅₀, C₂₅-C₁₇₅, or C₂₅-C₂₀₀ very long chain fatty acyl-CoA, very long chain fatty aldehyde, very long chain fatty alcohol, very long chain fatty ester wax, very long chain alkane, or very long chain ketone. In yet further embodiments, a very long chain carbon compound composition comprises a majority (more than 50% w/w) of very long chain carbon compounds having carbon chain lengths ranging from C₂₅ to C₄₀, from C₂₅ to C₅₀, from C₂₅ to C₇₅, from C₂₅ to C₁₀₀, from C₂₅ to C₁₂₅, from C₂₅ to C₁₅₀, from C₂₅ to C₁₇₅, or from C₂₅ to C₂₀₀, or a majority of very long carbon chain compounds having carbon chain lengths of greater than C₂₅, or a very long carbon chain compound containing composition wherein at least 70% of the total very long chain carbon compounds comprises C₂₅ to C₅₀ very long carbon chain compound.

In further embodiments, a C₁ metabolizing non-photosynthetic microorganism very long carbon chain compound containing biomass or a very long carbon chain compound composition has a δ¹³C of less than about −30‰, or ranges from about −40‰ to about −60‰. In certain embodiments, the very long carbon chain compound containing biomass comprises a recombinant C₁ metabolizing non-photosynthetic microorganism together with the spent media, or the very long carbon chain compound containing biomass comprises a spent media supernatant composition from a culture of a recombinant C₁ metabolizing non-photosynthetic microorganism, wherein the δ¹³C of the very long carbon chain compound containing biomass or a very long carbon chain compound composition obtained therefrom is less than about −30‰. In certain other embodiments, a very long carbon chain compound composition is isolated, extracted or concentrated from a very long carbon chain compound containing biomass, which can comprise recombinant C₁ metabolizing non-photosynthetic microorganisms together with the spent media from a culture, or a spent media supernatant composition from a culture of a recombinant C₁ metabolizing non-photosynthetic microorganism.

In certain embodiments, very long carbon chain compound containing biomass or a very long carbon chain compound composition is of a recombinant C₁ metabolizing non-photosynthetic microorganism that comprises one or more elongase complex enzymes, as disclosed herein, codon optimized for efficient expression in a C₁ metabolizing non-photosynthetic microorganism.

Exemplary organisms for use in generating very long carbon chain compound containing biomass or a very long carbon chain compound composition is of a recombinant C₁ metabolizing non-photosynthetic microorganisms of this disclosure, such as bacteria. In certain embodiments, very long carbon chain compound containing biomass or a very long carbon chain compound composition is of a C₁ metabolizing bacteria from a methanotroph or methylotroph, such as a Methylomonas sp. 16a (ATCC PTA 2402), Methylosinus trichosporium OB3b (NRRL B-11,196), Methylosinus sporium (NRRL B-11,197), Methylocystis parvus (NRRL B-11,198), Methylomonas methanica (NRRL B-11,199), Methylomonas albus (NRRL B-11,200), Methylobacter capsulatus Y (NRRL B-11,201), Methylococcus capsulatus Bath (NCIMB 11132), Methylobacterium organophilum (ATCC 27,886), Methylomonas sp. AJ-3670 (FERM P-2400), Methylomicrobium alcaliphilum, Methylocella silvestris, Methylacidiphilum infernorum, Methylibium petroleiphilum, Methylobacterium extorquens, Methylobacterium radiotolerans, Methylobacterium populi, Methylobacterium chloromethanicum, Methylobacterium nodulans, or any combination thereof.

In further embodiments, a very long carbon chain compound containing biomass or a very long carbon chain compound composition is of a C₁ metabolizing bacteria from a recombinant C₁ metabolizing bacteria of this disclosure is a syngas metabolizing bacteria, such as Clostridium autoethanogenum, Clostridium ljungdahli, Clostridium ragsdalei, Clostridium carboxydivorans, Butyribacterium methylotrophicum, Clostridium woodii, Clostridium neopropanologen, or a combination thereof.

### EXAMPLES

### Example 1

### Lipid Extraction from C₁ Metabolizing Microorganisms

A fatty acid oil composition contained within a harvested bacterial biomass was extracted using a modified version of Folch's extraction protocol (Folch et al., J. Biol. Chem. 226:497, 1957), performed at 20° C. (i.e., room temperature) and in an extraction solution made up of one volume methanol in two volumes chloroform (CM solution). About 5 g wet cell weight (WCW) of either fresh bacterial biomass (or bacterial biomass stored at −80° C. and subsequently thawed) was used for extractions. A 100 mL CM solution was added to the cell material and the mixture was extracted vigorously in a separatory funnel. After at least 10 minutes, three phases were resolved. The organic phase containing extracted lipids settled at the bottom of the separatory funnel, which was drained into a clean glass bottle. The middle layer contained primarily lysed cellular materials and could be separated from the light water phase containing salts and other soluble cellular components.

Optionally, solids in the water phase can be concentrated using a centrifuge or other mechanical concentration equipment. The water removed from the solids may be recycled, while the solids, with some residual water, can be fed to a solids processing unit.

To enhance the lipid extraction efficiency, a second extraction step was carried out by adding an additional 100 mL fresh CM solution directly into the separatory funnel containing the remaining lysed cell mass and residual water. The mixture was again mixed thoroughly, the phases allowed to separate, and the bottom organic phases from the two extractions were pooled. The pooled organic phases were then washed with 100 mL deionized water in a separatory funnel to remove any residual water-soluble material. The separated organic fraction was again isolated from the bottom of the separatory funnel and solvent was removed by rotary evaporation with heat, preferably in the absence of oxygen, or by evaporation at 55° C. under a stream of nitrogen.

TABLE 1
Extracted Lipid Content from Three Different Methanotrophs

| | | Lipid Fraction |
|---|---|---|
| Batch No. | Reference Strain | (g/g DCW)* |
| 68C | Methylosinus trichosporium OB3b | 40.1 |
| 62A | Methylococcus capsulatus Bath | 10.3 |
| 66A | Methylomonas sp. 16a | 9.3 |

*Grams of extracted material per gram of dry cell weight (DCW)

The solidified fatty acid compositions extracted from the harvested cultures of M. trichosporium OB3b, Methylococcus capsulatus Bath, and Methylomonas sp. 16a were each weighed and are shown as the weight fraction of the original dry cell weight (DCW) in Table 1. These data show that a significant fraction of the DCW from these C₁ metabolizing microorganisms is made up of lipids.

The fatty acid composition from Methylomonas sp. 16a biomass was also extracted using hexane:isopropanol (HIP) extraction method of Hara and Radin (Anal. Biochem. 90:420, 1978). Analysis of the fatty acid composition extracted using the HIP method showed that the fatty acid composition was essentially identical to the fatty acid composition extracted using the modified Folch method (data not shown).

### Example 2

### Fatty Acid Methyl Ester Conversion of Lipids from C₁ Metabolizing Microorganisms

The lipid fractions extracted from M. capsulatus Bath, M. trichosporium OB3b, and Methylomonas sp. 16a culture biomass in the form of dry solids were individually hydrolyzed with potassium hydroxide (KOH) and converted into fatty acid methyl esters (FAMEs) via reaction with methanol in a single step. About 5 g of extracted solid lipids in a 10 mL glass bottle were dissolved with 5 mL of 0.2 M KOH solution of toluene:methanol (1:1 v/v). The bottle was agitated vigorously and then mixed at 250 rpm at 42° C. for 60 minutes, after which the solution was allowed to cool to ambient temperature and transferred to a separatory funnel. Approximately 5 mL distilled water and 5 mL CM solution were added to the separatory funnel, mixed, and then the phases were allowed to separate by gravity or by centrifugation (3,000 rpm, 25° C.) for 5 minutes. The top aqueous layer was removed, which contains dissolved glycerol phosphate esters, while the heavy oil phase (bottom) was collected and concentrated to dryness by rotary evaporation or by a constant nitrogen stream.

Analysis of FFAs and FAMEs found in lipids from each methanotroph culture was performed using a gas chromatograph/mass spectrometer (GC/MS). The solids collected before and after the hydrolysis/transesterification step were dissolved in 300 μL butyl acetate containing undecanoic acid as an internal standard for GC/MS analysis. The resulting solution was centrifuged for 5 minutes at 14,000 rpm to remove insoluble residues. The same volume equivalent of N,O-Bis(trimethylsilyl)trifluoroacetamide was added to the supernatant from the centrifugation step and vortexed briefly. Samples were loaded on an GC equipped with mass spectrometer detector (HP 5792), and an Agilent HP-5MS GC/MS column (30.0 m×250 μm×0.25 μm film thickness) was used to separate the FFAs and FAMEs. Identity of FFAs and FAMEs was confirmed with retention time and electronionization of mass spectra of their standards. The GC/MS method utilized helium as the carrier gas at a flow of 1.2 mL/min. The injection port was held at 250° C. with a split ratio of 20:1. The oven temperature was held at 60° C. for 1 minute followed by a temperature gradient comprising an 8° C. increase/min until 300° C. The % area of each FFA and FAME was calculated based on total ions from the mass detector response.

The solid residue collected before and after hydrolysis/transesterification were analyzed for FFAs and FAMEs by GC/MS (see Table 2).

TABLE 2
Relative composition of FFA and FAME in Extracted Lipids
Before and After KOH Hydrolysis/Esterification

| | M. capsulatus | | M. trichosporium | | Methylomonas | |
|---|---|---|---|---|---|---|
| | Bath | | OB3b | | sp. 16a | |
| | With | Without | With | Without | With | Without |
| Fatty | hydro- | hydro- | hydro- | hydro- | hydro- | hydro- |
| Acid | lysis | lysis | lysis | lysis | lysis | lysis |
| Type | % Area | | % Area | | % Area | |
| C14:0 | — | — | — | — | — | 12.9 |
| FFA | | | | | | |
| C16:0 | 0.5 | 84.1 | — | 43.7 | — | 8.1 |
| FFA | | | | | | |
| C16:1 | — | 13.4 | — | — | — | 76.1 |
| FFA | | | | | | |
| C18:0 | 0.4 | 2.5 | — | 31.2 | — | 1.3 |
| FFA | | | | | | |
| C18:1 | — | — | — | 25.1 | — | 1.5 |
| FFA | | | | | | |
| C14:0 | 3.4 | — | — | — | 7.2 | — |
| FAME | | | | | | |
| C16:0 | 54.4 | — | 1.4 | — | 14.7 | — |
| FAME | | | | | | |
| C16:1 | 41.3 | — | 6.8 | — | 61.3 | — |
| FAME | | | | | | |
| C18:0 | — | — | 1.0 | — | N.D. | — |
| FAME | | | | | | |
| C18:1 | — | — | 90.8 | — | 16.8 | — |
| FAME | | | | | | |

* — = Not detectable; % Area: MS detector response-Total ions

As is evident from Table 2, extracted lipid compositions before hydrolysis/transesterification have abundant free fatty acids and additional fatty acids present, but the FFAs are converted into fatty acid methyl esters of various lengths after hydrolysis/transesterification. These data indicate that oil compositions from the C₁ metabolizing microorganisms of this disclosure can be refined and used to make high-value molecules.

### Example 3

### Stable Carbon Isotope Distribution in Lipids from C₁ Metabolizing Microorganisms

Dry samples of M. trichosporium biomass and lipid fractions were analyzed for carbon and nitrogen content (% dry weight), and carbon (¹³C) and nitrogen (¹⁵N) stable isotope ratios via elemental analyzer/continuous flow isotope ratio mass spectrometry using a CHNOS Elemental Analyzer (vario ISOTOPE cube, Elementar, Hanau, Germany) coupled with an IsoPrime100 IRMS (Isoprime, Cheadle, UK). Samples of methanotrophic biomass cultured in fermenters or serum bottles were centrifuged, resuspended in deionized water and volumes corresponding to 0.2-2 mg carbon (about 0.5-5 mg dry cell weight) were transferred to 5×9 mm tin capsules (Costech Analytical Technologies, Inc., Valencia, Calif.) and dried at 80° C. for 24 hours. Similarly, previously extracted lipid fractions were suspended in chloroform and volumes containing 0.1-1.5 mg carbon were transferred to tin capsules and evaporated to dryness at 80° C. for 24 hours. Standards containing 0.1 mg carbon provided reliable δ¹³C values.

The isotope ratio is expressed in “delta” notation (‰), wherein the isotopic composition of a material relative to that of a standard on a per million deviation basis is given by δ¹³C (or δ¹⁵N)=(R_{Sample}/R_{Standard-1})×1,000, wherein R is the molecular ratio of heavy to light isotope forms. The standard for carbon is the Vienna Pee Dee Belemnite (V-PDB) and for nitrogen is air. The NIST (National Institute of Standards and Technology) proposed SRM (Standard Reference Material) No. 1547, peach leaves, was used as a calibration standard. All isotope analyses were conducted at the Center for Stable Isotope Biogeochemistry at the University of California, Berkeley. Long-term external precision for C and N isotope analyses is 0.10‰ and 0.15‰, respectively.

M. trichosporium strain OB3b was grown on methane in three different fermentation batches, M. capsulatus Bath was grown on methane in two different fermentation batches, and Methylomonas sp. 16a was grown on methane in a single fermentation batch. The biomass from each of these cultures was analyzed for stable carbon isotope distribution (δ¹³C values; see Table 3).

TABLE 3
Stable Carbon Isotope Distribution in Different Methanotrophs

| Methanotroph | Batch No. | EFT (h)† | OD₆₀₀ | DCW* | δ¹³C Cells |
|---|---|---|---|---|---|
| Mt OB3b | 68A | 48 | 1.80 | 1.00 | −57.9 |
| | | 64 | 1.97 | 1.10 | −57.8 |
| | | 71 | 2.10 | 1.17 | −58.0 |
| | | 88 | 3.10 | 1.73 | −58.1 |
| | | 97 | 4.30 | 2.40 | −57.8 |
| | | 113 | 6.00 | 3.35 | −57.0 |
| | | 127 | 8.40 | 4.69 | −56.3 |
| Mt OB3b | 68B | 32 | 2.90 | 1.62 | −58.3 |
| | | 41 | 4.60 | 2.57 | −58.4 |
| | | 47 | 5.89 | 3.29 | −58.0 |
| | | 56 | 7.90 | 4.41 | −57.5 |
| Mt OB3b | 68C | 72 | 5.32 | 2.97 | −57.9 |
| | | 79.5 | 5.90 | 3.29 | −58.0 |
| | | 88 | 5.60 | 3.12 | −57.8 |
| | | 94 | 5.62 | 3.14 | −57.7 |
| Mc Bath | 62B | 10 | 2.47 | 0.88 | −59.9 |
| | | 17.5 | 5.80 | 2.06 | −61.0 |
| | | 20 | 7.32 | 2.60 | −61.1 |
| | | 23 | 9.34 | 3.32 | −60.8 |
| | | 26 | 10.30 | 3.66 | −60.1 |
| Mc Bath | 62A | 10 | 2.95 | 1.05 | −55.9 |
| | | 13.5 | 3.59 | 1.27 | −56.8 |
| | | 17.5 | 5.40 | 1.92 | −55.2 |
| | | 23 | 6.08 | 2.16 | −57.2 |
| | | 26 | 6.26 | 2.22 | −57.6 |
| Mms 16a | 66B | 16 | 2.13 | 0.89 | −65.5 |
| | | 18 | 2.59 | 1.09 | −65.1 |
| | | 20.3 | 3.62 | 1.52 | −65.5 |
| | | 27 | 5.50 | 2.31 | −66.2 |
| | | 40.5 | 9.80 | 4.12 | −66.3 |

*DCW, Dry Cell Weight is reported in g/L calculated from the measured optical densities (OD₆₀₀) using specific correlation factors relating OD of 1.0 to 0.558 g/L for Mt OB3b, OD of 1.0 to 0.355 g/L for Mc Bath, and OD of 1.0 to 0.42 g/L for Mms 16a. For Mt OB3b, the initial concentration of bicarbonate used per fermentation was 1.2 mM or 0.01% (Batch No. 68C) and 0.1% or 12 mM (Batch Nos. 68A and 68B).
†EFT = effective fermentation time in hours

In addition, stable carbon isotope analysis was performed for biomass and corresponding lipid fractions (see Table 4) from strains Methylosinus trichosporium OB3b (Mt OB3b), Methylococcus capsulatus Bath (Mc Bath), and Methylomonas sp. 16a (Mms 16a) grown on methane in bioreactors.

TABLE 4
Stable Carbon Isotope Distribution in Cells and Lipids

| Batch No. | Strain | δ¹³C Cells | δ¹³C Lipids |
|---|---|---|---|
| 68C | Mt OB3b | −57.7 | −48.6 |
| 62A | Mc Bath | −57.6 | −52.8 |
| 66A | Mms 16a | −64.4 | −42.2 |

Biomass from strains Mt OB3b, Mc Bath and Mms 16a were harvested at 94 h (3.14 g DCW/L), 26 h (2.2 g DCW/L) and 39 h (1.14 g DCW/L), respectively. The δ¹³C values for lipids in Table 4 represent an average of duplicate determinations.

### Example 4

### Effect of Methane Source and Purity on Stable Carbon Isotope Distribution in Lipids

To examine methanotroph growth on methane containing natural gas components, a series of 0.5-liter serum bottles containing 100 mL defined media MMS1.0 were inoculated with Methylosinus trichosporium OB3b or Methylococcus capsulatus Bath from a serum bottle batch culture (5% v/v) grown in the same media supplied with a 1:1 (v/v) mixture of methane and air. The composition of medium MMS1.0 was as follows: 0.8 mM MgSO₄*7H₂O, 30 mM NaNO₃, 0.14 mM CaCl₂, 1.2 mM NaHCO₃, 2.35 mM KH₂PO₄, 3.4 mM K₂HPO₄, 20.7 μM Na₂MoO₄*2H₂O, 6 μM CuSO₄*5H₂O, 10 μM Fe^{III}-Na-EDTA, and 1 mL per liter of a trace metals solution (containing, per L: 500 mg FeSO4*7H₂O, 400 mg ZnSO₄*7H₂O, 20 mg MnCl₂*7H2O, 50 mg CoCl₂*6H₂O, 10 mg NiCl₂*6H₂O, 15 mg H₃BO₃, 250 mg EDTA). Phosphate, bicarbonate, and Fe^{III}-Na-EDTA were added after media was autoclaved and cooled. The final pH of the media was 7.0±0.1.

The inoculated bottles were sealed with rubber sleeve stoppers and injected with 60 mL methane gas added via syringe through sterile 0.45 μm filter and sterile 27 G needles. Duplicate cultures were each injected with 60 mL volumes of (A) methane of 99% purity (grade 2.0, Praxair through Alliance Gas, San Carlos, Calif.), (B) methane of 70% purity representing a natural gas standard (Sigma-Aldrich; also containing 9% ethane, 6% propane, 3% methylpropane, 3% butane, and other minor hydrocarbon components), (C) methane of 85% purity delivered as a 1:1 mixture of methane sources A and B; and (D)>93% methane (grade 1.3, Specialty Chemical Products, South Houston, Tex.; in-house analysis showed composition>99% methane). The cultures were incubated at 30° C. (M. trichosporium strain OB3b) or 42° C. (M. capsulatus Bath) with rotary shaking at 250 rpm and growth was measured at approximately 12 hour intervals by withdrawing 1 mL samples to determine OD₆₀₀. At these times, the bottles were vented and headspace replaced with 60 mL of the respective methane source (A, B, C, or D) and 60 mL of concentrated oxygen (at least 85% purity). At about 24 hour intervals, 5 mL samples were removed, cells recovered by centrifugation (8,000 rpm, 10 minutes), and then stored at −80° C. before analysis.

Analysis of carbon and nitrogen content (% dry weight), and carbon (¹³C) and nitrogen (¹⁵N) stable isotope ratios, for methanotrophic biomass derived from M. trichosporium strain OB3b and M. capsulatus Bath were carried out as described in Example 3. Table 5 shows the results of stable carbon isotope analysis for biomass samples from M. capsulatus Bath grown on methane having different levels of purity and in various batches of bottle cultures.

TABLE 5
Stable Carbon Isotope Distribution of M. capsulatus Bath
Grown on Different Methane Sources having Different Purity

| Methane* | Batch No. | Time (h)† | OD₆₀₀ | DCW (g/L) | δ¹³C Cells |
|---|---|---|---|---|---|
| A | 62C | 22 | 1.02 | 0.36 | −40.3 |
| | | 56 | 2.01 | 0.71 | −41.7 |
| | | 73 | 2.31 | 0.82 | −42.5 |
| | 62D | 22 | 1.14 | 0.40 | −39.3 |
| | | 56 | 2.07 | 0.73 | −41.6 |
| | | 73 | 2.39 | 0.85 | −42.0 |
| B | 62E | 22 | 0.47 | 0.17 | −44.7 |
| | | 56 | 0.49 | 0.17 | −45.4 |
| | | 73 | 0.29 | 0.10 | −45.4 |
| | 62F | 22 | 0.62 | 0.22 | −42.3 |
| | | 56 | 0.63 | 0.22 | −43.6 |
| | | 73 | 0.30 | 0.11 | −43.7 |
| C | 62G | 22 | 0.70 | 0.25 | −40.7 |
| | | 56 | 1.14 | 0.40 | −44.8 |
| | | 73 | 1.36 | 0.48 | −45.8 |
| | 62H | 22 | 0.62 | 0.22 | −40.9 |
| | | 56 | 1.03 | 0.37 | −44.7 |
| | | 73 | 1.23 | 0.44 | −45.9 |

*Methane purity: A: 99% methane, grade 2.0 (min. 99%); B: 70% methane, natural gas standard (contains 9% ethane, 6% propane, 3% methylpropane, 3% butane); C: 85% methane (1:1 mix of A and B methane)
†Time = bottle culture time in hours

The average δ¹³C for M. capsulatus Bath grown on one source of methane (A, 99%) was −41.2±1.2, while the average δ¹³C for M. capsulatus Bath grown on a different source of methane (B, 70%) was −44.2±1.2. When methane sources A and B were mixed, an intermediate average δ¹³C of −43.8±2.4 was observed. These data show that the δ¹³C of cell material grown on methane sources A and B are significantly different from each other due to the differences in the δ¹³C of the input methane. But, cells grown on a mixture of the two gasses preferentially utilize ¹²C and, therefore, show a trend to more negative δ¹³C values.

A similar experiment was performed to examine whether two different methanotrophs, Methylococcus capsulatus Bath and Methylosinus trichosporium OB3b, grown on different methane sources and in various batches of bottle cultures showed a difference in δ¹³C distribution (see Table 6).

TABLE 6
Stable Carbon Isotope Distribution of Different Methanotrophs
Grown on Different Methane Sources of Different Purity

| | Meth- | Batch | Time | | DCW | δ¹³C |
|---|---|---|---|---|---|---|
| Strain | ane* | No. | (h)† | OD₆₀₀ | (g/L) | Cells |
| Mc | A | 62I | 18 | 0.494 | 0.18 | −54.3 |
| Bath | | | 40 | 2.33 | 0.83 | −42.1 |
| | | | 48 | 3.08 | 1.09 | −37.1 |
| Mc | D | 62J | 18 | 0.592 | 0.21 | −38.3 |
| Bath | | | 40 | 1.93 | 0.69 | −37.8 |
| | | | 48 | 2.5 | 0.89 | −37.8 |
| Mc | D | 62K | 18 | 0.564 | 0.20 | −38.6 |
| Bath | | | 40 | 1.53 | 0.54 | −37.5 |
| | | | 48 | 2.19 | 0.78 | −37.6 |
| Mt | A | 68D | 118 | 0.422 | 0.24 | −50.2 |
| OB3b | | | 137 | 0.99 | 0.55 | −47.7 |
| | | | 162 | 1.43 | 0.80 | −45.9 |
| Mt | A | 68E | 118 | 0.474 | 0.26 | −49.9 |
| OB3b | | | 137 | 1.065 | 0.59 | −47.6 |
| | | | 162 | 1.51 | 0.84 | −45.2 |
| Mt | D | 68F | 118 | 0.534 | 0.30 | −45.6 |
| OB3b | | | 137 | 1.119 | 0.62 | −38.7 |
| | | | 162 | 1.63 | 0.91 | −36.4 |
| Mt | D | 68G | 118 | 0.544 | 0.30 | −44.8 |
| OB3b | | | 137 | 1.131 | 0.63 | −39.1 |
| | | | 162 | 1.6 | 0.89 | −34.2 |

*Methane sources and purity: A: 99% methane (grade 2.0); D: >93% methane (grade 1.3)
†Time = bottle culture time in hours

The average δ¹³C for M. capsulatus grown on a first methane source (A) was −44.5±8.8, while the average δ¹³C for M. trichosporium was −47.8±2.0 grown on the same methane source. The average δ¹³C for M. capsulatus grown on the second methane source (B) was −37.9±0.4, while the average δ¹³C for M. trichosporium was −39.8±4.5. These data show that the δ¹³C of cell material grown on a methane source is highly similar to the δ¹³C of cell material from a different strain grown on the same source of methane. Thus, the observed δ¹³C of cell material appears to be primarily dependent on the composition of the input gas rather than a property of a particular bacterial strain being studied.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. provisional patent application Ser. No. 61/994,042, filed May 15, 2014, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for making a very long carbon chain compound, the method comprising:
(a) culturing a C₁ metabolizing non-photosynthetic microorganism with a C₁ substrate feedstock, wherein the C₁ metabolizing non-photosynthetic microorganism comprises one or more heterologous nucleic acid molecules encoding one or more of the following enzymes:
(i) a β-ketoacyl-CoA synthase (KCS);
(ii) a β-ketoacyl-CoA reductase (KCR);
(iii) a β-hydroxy acyl-CoA dehydratase (HCD); and
(iv) an enoyl-CoA reductase (ECR);
wherein the C₁ metabolizing non-photosynthetic microorganism converts the C₁ substrate into a very long carbon chain compound; and
(b) recovering the very long carbon chain compound.

2. The method of claim 1, wherein the very long carbon chain compound is a very long chain fatty acyl-CoA.

3. The method of claim 1, wherein the C₁ metabolizing non-photosynthetic microorganism further comprises:
(a) a heterologous nucleic acid molecule that encodes a fatty alcohol forming acyl-CoA reductase (FAR) capable of forming a very long chain fatty alcohol, wherein the very long carbon chain compound is a very long chain fatty primary alcohol;
(b) a heterologous nucleic acid molecule that encodes a fatty acyl-CoA reductase capable of forming a very long chain fatty aldehyde and a heterologous nucleic acid molecule encoding an aldehyde reductase capable of forming a very long chain fatty alcohol, wherein the very long carbon chain compound is a very long chain fatty primary alcohol;
(c) a heterologous nucleic acid molecule encoding a fatty acyl-CoA reductase capable of forming a very long chain fatty aldehyde, wherein the very long carbon chain compound is a very long chain fatty aldehyde;
(d) a heterologous nucleic acid molecule encoding a fatty acyl-CoA reductase capable of forming a very long chain fatty aldehyde and a heterologous nucleic acid molecule encoding an aldehyde decarbonylase capable of forming a very long chain alkane, wherein the very long, carbon chain compound is a very long chain alkane;
(e) a heterologous nucleic acid molecule encoding a fatty acyl-CoA, reductase capable of forming a very long chain fatty aldehyde, a heterologous nucleic acid molecule encoding an aldehyde decarbonylase capable of forming a very long chain forming, a very long chain fatty secondary alcohol, and a heterologous nucleic acid molecule encoding an alcohol dehydrogenase capable of forming a very long chain ketone, wherein the very long carbon chain compound is a very long chain ketone; or
(f) a heterologous nucleic acid molecule encoding a fatty alcohol forming acyl-CoA reductase capable of forming a very long chain fatty alcohol and a heterologous nucleic acid molecule encoding an ester synthase capable of forming a very long chain fatty ester wax, wherein the very long carbon chain compound is a very long chain fatty ester wax; and/or
(g) a heterologous nucleic acid molecule encoding a thioesterase; and/or
(h) a heterologous nucleic acid molecule encoding an acyl-CoA synthetase.

4.-8. (canceled)

9. The method of claim 1, wherein:
(a) the KCS is CER6, Elo1, Fen1/Elo2, Sur4/Elo3, KCS1, or FDH;
(b) the KCR is Ybr159w, AYR1, GL8A, GL8B, or At1g67730;
(c) the HCD is PHS1, PAS2, or PAS2-1; and/or
(d) the ECR is CER10 or TSC13.

10.-12. (canceled)

13. The method of claim 3, wherein:
(a) the fatty alcohol forming acyl-CoA reductase of subpart (a) from claim 3 is FAR, CER4, or Maqu_2220;
(b) the aldehyde reductase of subpart (b) from claim 3 is an alcohol dehydrogenase, wherein the alcohol dehydrogenase is YqhD;
(c) the fatty acyl-CoA reductase of subparts (c) and/or (d) from claim 3 is ACR1 or CER3;
(d) the aldehyde decarbonylase of subpart (d) from claim 3 is CER1 or CER22;
(e) the alkane hydroxylase of subpart (e) from claim 3 is MAH1;
(f) the alcohol dehydrogenase of subpart (e) from claim 3 is MAH1; and/or
(d) the ester synthase of subpart (f) from claim 3 is WSD1.

14.-19. (canceled)

20. The method according to claim 1, wherein:
(a) the C₁ metabolizing non-photosynthetic microorganism is a bacterium;
(b) the C₁ metabolizing non-photosynthetic microorganism is a methanotroph;
(c) the metabolizing non-photosynthetic microorganism is a methylotroph;
(d) the C₁ metabolizing non-photosynthetic microorganism is selected from the group consisting of Methylomonas, Methylobacter, Methylococcus, Methylosinus, Methylocystis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, and Pseudomonas;
(e) the C₁ metabolizing non-photosynthetic microorganism is selected from the group consisting of Methylococcus capsulatus Bath, Methylosinus trichosporium OB3b, Methylomonas sp. 16a, Methylomicrobium alcaliphilum, Methylosinus sporium, Methylocystis parvus, Methylomonas methanica, Methylomonas albus, Methylobacter capsulatus, Methylobacterium organophilum, Methylomonas sp. AJ-3670, Methylocella silvestris, Methylacidiphilum infernorum, and Methylibium petroleiphilum, or high growth variants thereof;
(f) the C₁ metabolizing non-photosynthetic microorganism is selected from the group consisting of Methylobacterium extorquens, Methylobacterium radiotolerans, Methylobacterium populi, Methylobacterium chloromethanicum, and Methylobacterium nodulans;
(g) the C₁ metabolizing non-photosynthetic microorganism is selected from the group consisting of Clostridium, Moorella, Pyrococcus, Eubacterium, Desulfobacterium, Carboxydothermus, Acetogenium, Acetobacterium, Acetoanaerobium, Butyribaceterium, and Peptostreptococcus; or
(h) the C₁ metabolizing non-photosynthetic microorganism is selected from the group consisting of Clostridium autoethanogenum, Clostridium ljungdahli, Clostridium ragsdalei, Clostridium carboxydivorans, Butyribacterium methylotrophicum, Clostridium woodii, and Clostridium neopropanologen.

21.-26. (canceled)

27. The method according to claim 20, wherein the culture further comprises a heterologous bacterium.

28.-32. (canceled)

33. The method according to claim 1, wherein the C₁ metabolizing non-photosynthetic microorganism is an obligate C₁ metabolizing non-photosynthetic microorganism.

34. (canceled)

35. The method according to claim 3, wherein:
(a) the encoded thioesterase of subpart (g) from claim 3 is a tesA lacking a signal peptide, UcFatB or BTE; and/or
(b) the acyl-Co synthetase of subpart (h) from claim 3 is FadD, yng1, or FAA2.

36. The method according to claim 3, wherein endogenous thioesterase activity is reduced, minimal or abolished as compared to unaltered endogenous thioesterase activity.

37.-38. (canceled)

39. The method according to claim 3, wherein endogenous acyl-CoA synthetase activity is reduced, minimal or abolished as compared to unaltered endogenous acyl-CoA synthetase activity.

40. The method according to claim 1, wherein the C₁ metabolizing non-photosynthetic microorganism produces:
(a) a very long carbon chain compound comprising one or more C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, or C₁₈₁-C₂₀₀ chain compounds;
(b) a very long carbon chain compound comprising a C₂₅-C₅₀ chain compound; or
(c) a fatty alcohol comprising C₂₅ to C₅₀ fatty alcohol and the C₂₅ to C₅₀ fatty alcohols comprise at least 70% of the total fatty alcohol.

41.-44. (canceled)

45. The method according to claim 1, wherein the C₁ substrate is natural gas, unconventional natural gas, syngas, methane, methanol, formaldehyde, formic acid or a salt thereof, carbon monoxide, carbon dioxide, a methylamine, a methylthiol, or a methylhalogen.

46. (canceled)

47. The method according to claim 1, wherein the C₁ metabolizing non-photosynthetic microorganism is a methanotroph bacterium, the C₁ substrate is methane, and the bacteria are cultured under aerobic conditions.

48. The method according to claim 1, further comprising culturing a C₁ metabolizing non-photosynthetic microorganism in a controlled culturing unit.

49. The method according to claim 48, wherein the C₁ substrate is methane, methanol, formaldehyde, formic acid or a salt thereof, carbon monoxide, carbon dioxide, natural gas, unconventional natural gas, syngas, a methylamine, a methylthiol, or a methylhalogen.

50. The method according to claim 48, wherein the controlled culturing unit is a fermentor or bioreactor.

51. A methanotroph, comprising one or more heterologous nucleic acid molecules encoding one or more of the following enzymes:
(i) a β-ketoacyl-CoA synthase (KCS);
(ii) a β-ketoacyl-CoA reductase (KCR);
(iii) a β-hydroxy acyl-CoA dehydratase (HCD); and
(iv) an enoyl-CoA reductase (ECR);
wherein the methanotroph is capable of converting a C₁ substrate into a very long carbon chain compound selected from a very long chain fatty acyl-CoA, a very long chain fatty aldehyde, a very long chain fatty primary alcohol, a very long chain fatty ester wax, a very long chain alkane, a very long chain fatty secondary alcohol, a very long chain ketone, or any combination thereof.

52. The methanotroph according to claim 51, wherein:
(a) the KCS is CER6, Elo1, Fen1/Elo2, Sur4/Elo3, KCS1, or FDH;
(b) the KCR is Ybr159w, AYR1, GL8A, GL8B, or At1g67730;
(c) the HCD is PHS1, PAS2, or PAS2-1; and/or
(d) the ECR is CER10 or TSC13.

53. The methanotroph according to claim 51, wherein the non-natural methanotroph comprises heterologous nucleic acid molecules encoding at least two different KCS enzymes.

54.-56. (canceled)

57. The methanotroph according to claim 51, further comprising:
(a) a heterologous nucleic acid molecule encoding a fatty alcohol forming acyl-CoA reductase (FAR) capable of forming a very long chain fatty alcohol;
(b) a heterologous nucleic acid molecule that encodes a fatty acyl-CoA reductase capable of forming a very long chain fatty aldehyde and a heterologous nucleic acid molecule encoding an aldehyde reductase capable of forming a very long chain fatty alcohol;
(c) a heterologous nucleic acid molecule encoding a fatty acyl-CoA reductase capable of forming a very long chain fatty aldehyde;
(d) a heterologous nucleic acid molecule encoding a fatty acyl-CoA reductase capable of forming a very long chain fatty aldehyde and a heterologous nucleic acid molecule encoding an aldehyde decarbonylase capable of forming a very long chain alkane;
(e) a heterologous nucleic acid molecule encoding a fatty acyl-CoA reductase capable of forming a very long chain fatty aldehyde, a heterologous nucleic acid molecule encoding an aldehyde decarbonylase capable of forming a very long chain alkane, a heterologous nucleic acid molecule encoding an alkane hydroxylase capable of forming a very long chain fatty secondary alcohol, and a heterologous nucleic acid molecule encoding an alcohol dehydrogenase capable of forming a very long chain ketone; or
(f) a heterologous nucleic acid molecule encoding a fatty alcohol forming acyl-CoA reductase capable of forming a very long chain fatty alcohol and a heterologous nucleic acid molecule encoding an ester synthase capable of forming a very long chain fatty ester wax; and/or
(g) a heterologous nucleic acid molecule encoding a thioesterase; and/or
(h) a heterologous nucleic acid molecule encoding an acyl-CoA synthetase.

58. The methanotroph of claim 57, wherein:
(a) the fatty alcohol forming acyl-CoA reductase of subpart (a) from claim 57 is FAR, CER4, or Maqu_2220;
(b) the aldehyde reductase of subpart (b) from claim 57 is an alcohol dehydrogenase, wherein the alcohol dehydrogenase is YqhD;
(c) the fatty acyl-CoA reductase of subparts (c) and/or (d) from claim 57 is ACR1 or CER3;
(d) the aldehyde decarbonylase of subpart (d) from claim 57 is CER1 or CER22;
(e) the alkane hydroxylase of subpart (e) from claim 57 is MAH1;
(f) the alcohol dehydrogenase of subpart (e) from claim 57 is MAH1; and/or
(d) the ester synthase of subpart (f) from claim 57 is WSD1.

59.-72. (canceled)

73. The methanotroph according to claim 57, wherein:
(a) the encoded thioesterase of subpart (g) from claim 57 is a tesA lacking a signal peptide, UcFatB or BTE; and/or
(b) the acyl-CoA synthetase of subpart (h) from claim 57 is FadD, yng1, or FAA2.

74. The methanotroph according to claim 57, wherein endogenous thioesterase activity is reduced, minimal or abolished as compared to unaltered endogenous thioesterase activity.

75.-76. (canceled)

77. The methanotroph according to claim 57, wherein endogenous acyl-CoA synthetase activity is reduced, minimal or abolished as compared to unaltered endogenous acyl-CoA synthetase activity.

78. The methanotroph according to claim 51, wherein the methanotroph produces:
(a) a very long carbon chain compound comprising one or more C₂₅-C₃₀, C₃₁-C₄₀, C₄₁-C₆₀, C₆₁-C₈₀, C₈₁-C₁₀₀, C₁₀₁-C₁₂₀, C₁₂₁-C₁₄₀, C₁₄₁-C₁₆₀, C₁₆₁-C₁₈₀, or C₁₈₁-C₂₀₀ chain compounds;
(b) a very long carbon chain compound comprising a C₂₅-C₅₀ chain compound; or
(c) a fatty alcohol comprising C₂₅ to C₅₀ fatty alcohol and the C₂₅ to C₅₀ fatty alcohols comprise at least 70% of the total fatty alcohol.

79.-84. (canceled)

85. The methanotroph according to claim 51, wherein the methanotroph is selected from Methylococcus capsulatus Bath, Methylomonas 16a, Methylosinus trichosporium OB3b, Methylosinus sporium, Methylocystis parvus, Methylomonas methanica, Methylomonas albus, Methylobacter capsulatus, Methylobacterium organophilum, Methylomonas sp AJ-3670, Methylocella silvestris, Methylocella palustris, Methylocella tundrae, Methylocystis daltona strain SB2, Methylocystis bryophila, Methylocapsa aurea KYG, Methylacidiphilum infernorum, Methylibium petroleiphilum, Methylomicrobium alcaliphilum, or any combination thereof.
